SEQUENCE LISTING (21 sequences)

SEQ ID NO: 1 - DNA, 20 bp, Homo sapiens
gttcttgaacgtgcggacac

SEQ ID NO: 2 - DNA, 21 bp, Human
gctgctctcaacatgcgagtg

SEQ ID NO: 3 - DNA, 21 bp, Human
actggaccgcgctggcggagc

SEQ ID NO: 4 - DNA, 21 bp, Human
gcttgaacgtgcggacacagg

SEQ ID NO: 5 - DNA, 20 bp, Human
cgctgaggctccaggaccgc

SEQ ID NO: 6 - DNA, 21 bp, Human
cctgtctctgttcttgaacgc

SEQ ID NO: 7 - DNA, 21 bp, Human
ctggctgctctcaacatgcgc

SEQ ID NO: 8 - DNA, 21 bp, Human
gtgtctctgttcttgaacgtg

SEQ ID NO: 9 - DNA, 20 bp, Human
gaccctagtgctcgtcgccg

SEQ ID NO: 10 - DNA, 20 bp, Human
gggcgcgacgaccctagtgc

SEQ ID NO: 11 - DNA, 20 bp, Human
gctcgtcgccgtggcgccat

SEQ ID NO: 12 - DNA, 21 bp, Human
tagtgctcgtcgccgtggcgc

SEQ ID NO: 13 - DNA, 20 bp, Human
agtgctcgtcgccgtggcgc

SEQ ID NO: 14 - DNA, 21 bp, Human
gtgctcgtcgccgtggcgcca

SEQ ID NO: 15 - DNA, 20 bp, Human
gcagcatagtgagcccagaa

SEQ ID NO: 16 - DNA, 20 bp, Human
ctccgtggaagaactgtggc

SEQ ID NO: 17 - DNA, 18 bp, Human
ggcggctgtatccaacgc

SEQ ID NO: 18 - DNA, 18 bp, Human
ggagtcgtcctggaatgc

SEQ ID NO: 19 - DNA, 23 bp, Human
acctcgagaactgacaattatgc

SEQ ID NO: 20 - DNA, 21 bp, Human
gcacagggtggaacaagatgg

SEQ ID NO: 21 - DNA, 21 bp, Human
caccaccccaaaggtgaccgt

(19) United States
(12) Patent Application Publication
(10) Pub. No.: US 2020/0208108 A1
(43) Pub. Date: Jul. 2, 2020

(54) INTERFERON PRIMED PLASMACYTOID DENDRITIC CELLS

(71) Applicant: AARHUS UNIVERSITET, Aarhus (DK)

(72) Inventors: Martin ROELSGAARD JAKOBSEN, Risskov (DK); Anders LAUSTSEN, Aarhus V (DK)

(21) Appl. No.: 16/612,441

(22) PCT Filed: May 8, 2018
(86) PCT No.: PCT/EP2018/061870

§ 371 Date: Nov. 11, 2019

(30) Foreign Application Priority Data
May 10, 2017 (EP) 17170373.9
Jan. 8, 2018 (EP) 18150546.2

(51) Int. Cl. C12N 5/0784; A61K 35/15

(52) U.S. Cl. CPC C12N 5/0639; A61K 35/15; C12N 2506/11; C12N 2510/00; C12N 2501/24; C12N 2501/2303; C12N 2501/26; C12N 2501/125

(57) ABSTRACT

A method is provided for producing a plasmacytoid dendritic cells (pDCs), wherein hematopoietic stem and progenitor cells (HSPCs) are provided and incubated in a first medium comprising cytokines and growth factor whereby the HSPCs are differentiated into precursor-pDCs and then adding interferons (IFNs) to the first medium to obtain a second medium whereby said precursor-pDCs are differentiated into pDCs. Furthermore, a technique is provided for producing genetically modified pDCs, by initially genetically modifying HSPCs using transfection methods, including electroporation, to deliver sgRNA and Cas9 protein Moreover, a pharmaceutical formulation and a vaccine is provided which comprises pDC or genetically modified pDCs obtained according to that method.

### TECHNICAL FIELD

The present invention relates to a method for producing interferon primed plasmacytoid dendritic cells, antigen presenting cells and genetically engineered plasmacytoid dendritic cells, vaccines and pharmaceutical formulation comprising said plasmacytoid dendritic cells or antigen presenting cells and methods for treating infectious disease and/or cancer.

### BACKGROUND

Plasmacytoid dendritic cells (pDCs) are essential for immune competence, yet their development and functions remain elusive. pDCs are key effectors in cellular immunity with the ability to not only initiate immune responses but also to induce tolerance to exogenous and endogenous antigens (Swiecki, M. and M. Colonna, The multifaceted biology of plasmacytoid dendritic cells. Nat Rev Immunol, 2015. 15(8): p. 471-85). pDCs are distinct from conventional DCs as their final stage of development occurs within the bone marrow; their antigens are taken up by receptor-mediated endocytosis; they express high levels of interferon regulatory factor 7; and they primarily sense pathogens through toll-like receptor (TLR) 7 and 9 (Swiecki, M. and M. Colonna, The multifaceted biology of plasmacytoid dendritic cells. Nat Rev Immunol, 2015. 15(8): p. 471-85; and Tang, M., J. Diao, and M. S. Cattral, Molecular mechanisms involved in dendritic cell dysfunction in cancer. Cell Mol Life Sci, 2016). Through these pattern-recognition receptors, pathogen nucleic acids can activate pDCs to produce high levels of type I interferon (IFN). Thus, activated pDCs link the innate and adaptive immune system together via cytokine production combined with antigen-presenting cell (APC) activity. Furthermore, pDC functionality is also essential to achieve an antiviral state during infections, provide vital adjuvant activity in the context of vaccination, and for promoting immunogenic anti-tumor responses upon activation (Swiecki, M. and M. Colonna, The multifaceted biology of plasmacytoid dendritic cells. Nat Rev Immunol, 2015. 15(8): p. 471-85; Tovey, M. G., C. Lallemand, and G. Thyphronitis, Adjuvant activity of type I interferons. Biol Chem, 2008. 389(5): p. 541-5; and Rajagopal, D., C. Paturel, Y. Morel, S. Uematsu, S. Akira, and S. S. Diebold, Plasmacytoid dendritic cell-derived type I interferon is crucial for the adjuvant activity of Toll-like receptor 7 agonists. Blood, 2010. 115(10): p. 1949-57). A delicate balance must be maintained, however, as hyper-activation of pDCs has been associated with the pathogenesis of several diseases, including viral infections, autoimmune diseases and tumorigenesis (Swiecki, M. and M. Colonna, The multifaceted biology of plasmacytoid dendritic cells. Nat Rev Immunol, 2015. 15(8): p. 471-85; and Tang, M., J. Diao, and M. S. Cattral, Molecular mechanisms involved in dendritic cell dysfunction in cancer. Cell Mol Life Sci, 2016). Collectively, pDCs have a key and multifaceted role in the immune system, prompting intense research into their development and function.

However, the low frequencies of pDCs within peripheral blood (less than 0.1% of peripheral blood mononuclear cells) and their poor survival in cell culture environments, has severely limited progress in understanding pDC biology (Ueda, Y., M. Hagihara, A. Okamoto, A. Higuchi, A. Tanabe, K. Hirabayashi, S. Izumi, T. Makino, S. Kato, and T. Hotta, Frequencies of dendritic cells (myeloid DC and plasmacytoid DC) and their ratio reduced in pregnant women: comparison with umbilical cord blood and normal healthy adults. Hum Immunol, 2003. 64(12): p. 1144-51; and Zhan, Y., K. V. Chow, P. Soo, Z. Xu, J. L. Brady, K. E. Lawlor, S. L. Masters, M. O'Keeffe, K. Shortman, J. G. Zhang, and A. M. Lew, Plasmacytoid dendritic cells are short-lived: reappraising the influence of migration, genetic factors and activation on estimation of lifespan. Sci Rep, 2016. 6: p. 25060). Consequently, the availability of a simple, flexible and reproducible pDC culture system would facilitate a rapid elucidation of pDC development as well as the recapitulation of immune functions essential for understanding the biology of pDCs in the innate immune system. This has also tremendous potential regarding the use of pDCs in immunotherapeutic methods, including the development of new medicaments, in particular vaccines, as well as methods for preventing and/or treating cancers and/or infectious diseases. Thus, developing therapies that restore pDC functionality and trigger innate activation to produce type I IFN can be a valuable tool to induce effective anti-tumor immunity.

Production of pDC has been described in the prior art. For example Demoulin et al (Exp Hematol. 2012 April; 40(4):268-78) discloses culturing human CD34+ hematopoietic cells isolated from cord blood in the presence of TPO, Flt3L and one of the cytokines: IL-3, IFN-beta or PGE2. It is reported that the combination of TPO, Flt3L and IL-3 yields high levels of pDCs, which are CD304+, CD123+CD11c+. Thordardottir et al (Stem Cells Dev. 2014 May 1; 23(9):955-67) describes CD34+ cells derived from umbilical cord blood cultured in a medium comprising TPO, SCF, Tlt3L and IL-6. Cells are reported to differentiate into pDCs secreting high levels of pro-inflammatory cytokines, such as INF-a, IL-12 and TNF-a. There is, however, no disclosure of pDCs secreting high levels of pro-inflammatory cytokines, such as IFN-a, IL-12 and TNF-a, in response to e.g. TLR7 agonists. Thordardottir et al (Oncoimmunology. 2017 Feb. 6; 6(3):e1285991) reports culturing CD34+ HSPCs in a medium supplemented with SR1 and Ftl3L, SCF and TPO in order to generate pDCs capable of activating NK cells. Olivier et al (Blood. 2006 Apr. 1; 107(7):2694-701. Epub 2005 Dec. 15.) describes culturing umbilical cord progenitor cells for generation of pDCs, which are CD303+, CD123+, CD4+, CD13c+. It is, however, a major limitation that this system is dependent on the co-culture of umbilical cord progenitor cells with a cancer stromal cell line in order to generate pDCs, which is not suitable for therapeutic purposes. Furthermore, the method does not produce sufficient numbers of pDCs. FR2848565 describes methods for production of pDC cell lines, which are CD4+, HLA DR+, CD123+, CD45 RA+, CD11c− et CD13−. However, this method is based on a cell line derived from one person with a specific HLA genotype, meaning that patients that should receive this therapy needs to be HLA-matched to that specific person to avoid potential allogeneic responses. A major limitation of all of these disclosures in the prior art is the lack of comparison of cytokine responses, such as TLR7 and TLR9, of the generated cells to natural pDCs, including TLR7 and TLR9 responses. Generally, the prior art disclosures also fails to compare the generated pDCs functionally and phenotypically to natural pDCs.Thus, methods for the production of large amounts of pDCs, accompanied by comparative evidence of the generated pDCs being similar functionally and phenotypically, i.e. proving their true similarity, has not been disclosed in the prior art.

In contrast to the disclosures of the prior art, the present technology offers a method of producing high numbers of pDCs from hematopoietic cells, which has been found to require additional factors in the form of interferons to reach a comparable function as natural pDCs, which is a hall mark of the present disclosure. Moreover, as the present technique is based on primary hematopoieitic cell stem cells, the method allows generating pDCs that are already HLA-matched to the patient (patient-specific cells), which makes the present technique superior for therapeutic purposes. In addition, the pDCs generated using the present technique are amenable to genetic modifications, allowing the generation a unique product of genetically altered pDCs for general studies or therapeutic purpose.

### SUMMARY

The inventors of the present invention have developed a highly efficient methodology for production of large amounts of activated pDCs with high viability. The inventors have found that the activated pDCs express the killing receptor, TRAIL, which is believed to induce killing of cancer cells. The inventors have also found that the activated pDCs function as APCs and thus are capable to take-up and present antigens that allows presentation to T-cells and trigger their activation. More specifically, the present invention relates to a culture method whereby hematopoietic progenitor cells (HSPCs) are expanded ex vivo and subsequently differentiated into mature pDCs in the presence of interferons. Thus, the inventors have found that a second culture condition that include interferon treatment can prime pDC precursors to develop into mature pDC with a phenotype strongly resembling that of blood pDCs. This two-step approach for generation of blood pDCs, where the HSPCs are pre-cultured in a first medium with cytokines and growth factors before being exposed to interferons has proven highly efficient in generating large amounts of mature pDCs. Indeed the provided method is highly superior in terms of yielding mature pDCs to one-step methods, where precursor cells are incubated with cytokines and/or growth factors together with interferon. For comparison, reference is made to example 1, herein below.

Moreover, the inventors have found that the pDCs are amenable to genetic modifications by initially modifying the HSPCs before generation of pDCs, allowing the production of pDCs with increased therapeutic potential.

Accordingly, the method of the present invention provides a powerful strategy for expanding and differentiating human HSPCs into pDCs that overcomes the great need for a robust ex vivo pDC culture system in for example treatment of cancer. The power in this model is found within its simplicity, the increased survival of the pDC and the high cell yields.

Thus, in one aspect the present invention relates to a method for producing a plasmacytoid dendritic cells (pDCs), said method comprising
providing hematopoietic stem progenitor cells (HSPCs)
incubating said HSPCs in a first medium comprising cytokines and growth factor whereby said HSPCs are differentiated into precursor-pDCs
adding interferons (IFNs) to said first medium to obtain a second medium
   whereby said precursor-pDCs are differentiated into pDCs

In a related aspect, a method is provided for producing a plasmacytoid dendritic cells (pDCs), said method comprising
providing hematopoietic stem progenitor cells (HSPCs)
incubating said HSPCs in a first medium comprising cytokines and growth factor whereby said HSPCs are differentiated into precursor-pDCs
adding SCF and SR1 in a first medium to obtain high yield of pre-cursor pDCs
providing a second medium comprising interferons (IFNs)
adding interferons (IFNs) to said a second medium to said first medium comprising pre-cursor pDCs, whereby said precursor-pDCs are transformed into to obtain a high yield of fully activated and differentiated pDCs a second medium
   whereby said precursor-pDCs are differentiated into pDCs

In a preferred embodiment said second medium comprises IFN-γ and/or IFN-β. In another embodiment said second medium further comprises IL-3. Preferably, said second medium comprises IL-3, IFN-γ and IFN-β.

The precursor-pDCs may for example be incubated for at least 24 hours in said second medium. Preferably, said precursor-pDCs are incubated for 24 to 72 hours in said second medium.

In one embodiment said first medium comprises Flt3 ligand, thrombopoietin and/or interleukin-3. In another embodiment said first medium further comprises stem cell factor and StemRegenin 1. In another embodiment said first medium further comprises stem cell factor and UM 171. In another embodiment said first medium further comprises RPMI medium supplemented with fetal calf serum (FCS). In another embodiment said first medium comprises serum-free medium (SFEM). Preferably, said first medium comprises Flt3 ligand, thrombopoietin, interleukin-3 and StemRegenin 1.

The HSPCs may for example be incubated for 21 days in said first medium.

In one embodiment of the present invention, incubation of said HSPCs in said first medium lead to an average yield of 60-100 precursor-pDCs per HSPC.

In one embodiment the method as described herein, further comprises a step of immunomagnetic negative selection to enrich for differentiated pDCs.

In one particular embodiment, the method of the present invention further comprises a step of incubating said pDCs with at least one antigen leading to the formation of antigen presenting cells (APCs).

In one embodiment said antigen is a cancer specific antigen. In another embodiment said antigen is a pathogen specific antigen. In yet another embodiment said antigen is a virus-, bacteria- or parasite specific antigen.

Preferably, said pDCs express TRAIL. In another embodiment said pDCs express CD123, CD303, CD304, CD4 and/or HLA-DR. In yet another embodiment said pDCs express IFN type I, IFN type II, IFN type III and/or proinflammatory cytokines. In a further embodiment said pDCs express IRF7, TLR7 and/or TLR9. In one embodiment said pDCs express CD40, CD80, CD83 and/or CD86. For example, said pDCs express TRAIL, CD123, CD303, CD304, CD4, HLA-DR, IFN type I, IFN type II, IFN type III, IRF7, TLR7 and/or TLR9.

In a preferred embodiment said pDCs secretes IL-6.

Another aspect of the present invention relates to a pDC and/or an APC population obtained by the method as described herein. The pDCs and/or an APCs of these populations are as defined herein.

Another aspect of the present invention relates to a pDC and/or APC population as defined herein for use in the treatment of an infectious disease or cancer.

A further aspect of the present invention relates to a pharmaceutical formulation, which comprises a pDC and/or an APC population and a pharmaceutically acceptable carrier.

The present invention also provides a vaccine comprising an immunologically effective amount of a pDC and/or an APC population as defined herein.

The present invention further provides a kit comprising a pDC and/or APC population as defined herein, and elements associated with immunological use. In one embodiment, the kit accordingly further comprises at least one antigen. Preferably said antigen is as defined elsewhere herein.

A further aspect of the present invention relates to a method of treating, preventing and/or ameliorating an infectious disease and/or cancer, said method comprising administering a therapeutically efficient amount of a pDC and/or APC population as obtained by the method of the present invention to an individual in need thereof.

Said infectious disease may for example be a viral infection or a bacterial infection. In one embodiment said cancer is TRAIL specific cancer.

Preferably, said pDCs and/or APCs are administered to the individual in need thereof by injection.

A further aspect of the present invention relates to a method for generating pDCs that have been genetically modified by initially genetically modifying HSPC prior to pDC differentiation. In one embodiment said pDCs have been genetically modified to have increased anti-tumor response through knock-out of co-inhibitory receptors, including PD-L1 (CD274) or LILRA4 (CD85g). In another embodiment said genetically modified pDCs have increased anti-tumor response through increased expression of TRAIL.

### DESCRIPTION OF DRAWINGS

FIG. 1. Generation of pDCs from hematopoietic stem cells (HSPC-pDC). a) Graphical illustration of the generation of HSPC-pDCs from HSPCs. CD34⁺ HSPC were isolated from human umbilical cord blood (UCB). Briefly, mononucleated cells were recovered by Ficoll-Hypaque density-gradient centrifugation. CD34⁺ HSPCs were then isolated using anti-CD34 immunomagnetic beads. Next, 2'10⁵ CD34⁺ HSPC were cultivated for 21 days with factors promoting the expansion and differentiation of cells. At day 21 the cell suspension was magnetically enriched for lin^{neg}CD11c^{neg} pDCs using negative selection. b) Total numbers of suspension cells measured at the initial experiment start (day 1) and the consecutive culture days (4, 8, 12, 16, 18 and 21) with the indicated factors. c) Representative flow cytometry plot showing linage and CD11c markers before and after enrichment of pDCs. d) Total numbers (upper panel) or percentage yield (lower panel) of HSPC-pDCs generated after magnetic enrichment at day 21 from Flt3-L, TPO, IL-3 culture supplemented with indicated growth factors. Error bars represent ±SEM of four donors.

FIG. 2. Generation of HSC-pDCs from HSC. a) Flow cytometric analysis of CD34 surface expression on cord-blood derived mononuclear cells (CBMC) before and after isolaion of CD34+ cells. b) Yield of CD34+ HSC in total numbers (left) or per mL of umbilical cord blood (right). Data are ±SEM and each dot represent a single donor of 13 donors. c) May-Grünwald-Giemsa stain of cultured cells at 21 day before or after enrichment of HSC-pDCs.

FIG. 3. HSPC-pDCs elicit a precursor-pDCs phenotype. a) Phenotypic analysis of pDC-associated markers on HSPC-pDC at day 21 using Flow Cytometry. For gating strategy see Supplementary FIG. 2a-b b) Surface expression levels of pDC-related markers on enriched HSPC-pDCs at day 21 in comparison to blood pDCs. c) Graphical illustration of removal of growth factors of HSPC-pDC. After enrichment of HSPC-pDCs at day 21, cells were depleted of growth factors (except IL-3). After 24 hours (day 21+1) HSPC-pDCs were harvested for phenotypic or functional analyses. d) Phenotypic comparison of HSPC-pDCs at day 21 or HSPC-pDCs at day 21+1. e, f) Levels of type I IFN (e) or IL-6 (f) after stimulation with TLR7 or TLR9 agonists of enriched HSPC-pDCs cells at day 21, HSPC-pDCs Day 21+1^{IL-3} or blood pDCs. Data shown are ±SEM of three donors.

FIG. 4. Gating strategy of HSC-pDCs and blood pDCs. a) Gating strategy to discriminate HSC-pDCs. b) Phenotypic analysis of CD123, CD303, CD304, HLA-DR and CD4. Cells were first gated based on FSC and SSC to exclude debris, and then doublets were excluded in regards to FSC-A and SSC-A. A viability stain, a lineage stain (lin) and a CD11c stain was next used to gate on viable/linneg/CD11cneg cells for the indicated pDC-related markers. Fluorescence minus one (FMO) controls were utilized to set the gates for the positive/negative events. c) Phenotypic analysis of blood pDCs with the same gates as determined in b).

FIG. 5. Removal of growth factors promote the up-regulation of pDC specific markers on HSC-pDCs. Expression levels of CD123, CD303, CD304, HLA-DR or CD4 on HSC-pDCs at day 21 or after one day of culture in medium depleted for growth factors (day 21+1^{IL-3}) for all tested growth conditions. Levels are either depicted as percentages of all viable cells (a) or as absolute MFI values (b). c) Representative histograms showing the expression of CD123, CD303, CD304, CD4 and HLA on HSC-pDCs at day 21 and day 21+1^{IL-3}. Data are ±SEM of four donors.

FIG. 6. Upon priming HSPC-pDCs can be modulated to become classical pDCs. a) Graphical illustration of HSPC-pDC priming. After enrichment, HSPC-pDCs were primed in medium supplemented with IFN-β+γ or IL-3 alone. After 72 hours (day 21+3) primed cells were harvested for phenotypic or functional analyses. b) Surface expression levels of pDC-related markers on HSPC-pDC cells after priming for 72 hours with IFN-β+γ (Day 21+3^{IFN β+γ}) and blood pDCs. c, d) Type I IFN (c) or IL-6 (d) response of blood pDCs, non-primed or primed HSPC-pDCs cells to TLR7 and TLR9 agonists. e) Type I IFN response of HSPC-pDCs primed with titrated concentrations of IFN-β+γ before being stimulated with the TLR7 agonist R837. Data shown are ±SEM of three donors.

FIG. 7. Priming of HSC-pDCs up-regulates the surface expression of pDC-related markers. a) Phenotypic analysis of HSC-pDCs at day 21+1 or day 21+3 under indicated priming conditions. b) Phenotypic comparison of HSC-pDCs at day 21, IFN-β+γ primed HSC-pDCs at day 21+3 and blood pDCs. c, d) Phenotypic comparison of different priming condition on HSC-pDCs, either depicting the percentage of positive cells (c) or absolute MFI values (d). Each bar shows the mean of five donors±SEM.

FIG. 8. Priming of HSC-pDCs increases the functional type I IFN response to TLR agonists. a, b) Type I IFN produced in response to TLR7 and TLR9 agonists of primed HSC-pDCs at day 21+1 (a) and day 21+3 (b). Each dot represents individual donors. Data are ±SEM of triplicates from three donors.

FIG. 9. Upon priming HSC-pDCs elicit an IL-6 response. a, b) Levels of IL-6 produced in response to TLR7 and TLR9 agonists of primed HSC-pDCs at day 21+1 (a) and day 21+3 (b). Each dot represents individual donors. Data are ±SEM of triplicates from three donors.

FIG. 10. Titration of Interferon on HSC-pDCs. HSC-pDCs were primed with varying concentrations of IFN-β/γ ranging from 1.95 to 500 U/mL or left non-primed (0 U/mL). After three days cells were phenotypically analyzed for the expression of CD123 and CD304 using flow cytometry or stimulated with R837 for functional validation of the type I IFN response. a, b) Phenotypic analysis of CD123 (left) or CD304 (right) of non-primed or HSC-pDCs primed with either 62.5, 250 or 500 U/mL of IFN-β/γ. c, d) Phenotypic analysis of HSC-pDCs at day 21+3 under indicated priming conditions. Column diagrams show percentage of CD123 or CD304 (c, d). e) Type I IFN produced in response to the TLR7 agonist R837 of primed HSC-pDCs at day 21+3 for each donor tested. Each dot represent individual donors. Data are ±SEM of singlets (a-d) or triplicates (e) of three donors.

FIG. 11. Expression levels of genes of primed and non-primed HSC-pDCs. HSC-pDCs were primed with IFN-β+γ or were left non-primed with IL-3 alone. After one day (day 21+1) or three days (day 21+3), cells were lysed and the gene expression level of IRF7 (a), TLR7 (b) or TLR9 (c) was assessed using qPCR. Data shown are ±SEM of biological triplicates from four donors, either shown as individual (right) or collective (left) donors.

FIG. 12. Expression levels of IFN-α2, IFN-α4 and IFN-α16 after resting phase of HSC-pDCs. a) Graphical illustration of resting of HSC-pDCs. Cells were primed or left non-primed. After 24 hours (Day 21+1) cells were either lysed or washed and resuspend in medium with IL-3 alone and cultivated for an additional 24 hours (rest) before being lysed. Basal expression levels of selected IFN-α genes was subsequently evaluated using qPCR. b) Basal expression levels of IFN-α4 and IFN-α16. Data are ±SEM of biological triplicates from two donors.

FIG. 13. HSPC-pDCs have the capacity to adopt an antigen presenting phenotype upon stimulation and demonstrate prolonged survival in comparison to blood pDCs. a) Expression levels of the indicated maturation markers on primed/non-primed HSPC-pDCs at day 21+3 after stimulation with the TLR7 agonist R837. b) Blood pDCs or HSPC-pDCs were cultivated for 1, 3, 5, 8 and 12 days in medium supplemented with IL-3. At each time-point the viability was assessed by flow cytometry. c) Comparison of area under curve of HSPC-pDCs and blood pDCs in the different conditions tested. Data shown are ±SEM of three (a) or four (b-c) donors.

FIG. 14. TLR stimulation of primed HSC-pDCs up-regulates co-stimulatory markers. a) Graphical illustration of the maturation process of HSC-pDCs. Cells were primed for three days (day 21+3) with IFN-β+γ or left non-primed (IL-3). Cells were then either phenotypically analyzed or stimulated for an additional 24 hours with the TLR7 agonist R837 before phenotypic characterization. b) Phenotypic analysis of CD40, CD80, CD83, CD86 and CD253 of non-primed or primed HSC-pDCs stimulated with R837 (lower) or left untreated (UT) (upper panel).

FIG. 15. Viability of HSC-pDCs and blood pDCs upon prolonged culture. a, b) HSC-pDCs and blood pDCs were cultivated for 1, 3, 5, 8 and 12 days in medium supplemented with IL-3 or IL-3+IFN-β/γ. At each time point number of viable cells were assessed using flow cytometry. c, d) Percentage of viable cells over prolonged culture calculated from panel (a) and (b). e, f) Area under curve calculated from (c) and (d). Data are ±SEM of biological duplicates (for blood pDCs) or triplicates (for HSC-pDCs) from four donors at each time-point.

FIG. 16. HSPC-pDCs are capable of presenting antigens and activating T cells a) Graphical illustration of the experiment setup for T cell stimulatory capacity of HSPC-pDCs. HSPC-pDCs (primed and activated by the TLR7 agonist R837) were pulsed with a mix of peptides derived from CMV peptides (CMV ProMix) or remained untreated (UT). After three hours cells were washed and co-cultured with PBMCs harvested from CMV seropositive donors and T cell activity was subsequently evaluated by measuring levels of IFN-γ in the supernatant 20 hours later. b) Levels of IFN-γ produced by PBMCs from three CMV seropositive donors after stimulation with CMV ProMix. c) Levels of IFN-γ produced by same three PBMC donors mixed in a 10:1 ratio with three HSPC-pDC donors (IFN primed and TLR7 activated) with or without a three hour pulse of CMV ProMix. Data shown are ±SEM of three (b-c).

FIG. 17. HSPC-pDCs can be generated using serum-free conditions. a) Numbers and percentage of HSPC-pDCs generated using serum-free conditions (SFEM II) or RPMI medium supplemented with FCS. b) Phenotypic comparison of primed and non-primed HSPC-pDCs (viable, lineage⁻CD11c⁻ cells) at day 21+3. Upper panel show HSPC-pDCs generated using RPMI medium supplemented with FCS and lower HSPC-pDCs generated using SFEM II. c) Type I IFN produced in response to TLR7 and TLR9 agonists of primed and non-primed HSPC-pDCs derived from HSPC cultured in SFEM II or RPMI medium supplemented with FCS. Data shown are from three donors (a), two donors (b) and two donors done in biological dublicates (c).

FIG. 18. HSPC-pDCs are amenable to genetic modifications. a) Graphical illustration of the protocol for CRISPR/Cas9-mediated gene editing using Cas9 RNP. CD34⁺ HSPCs were initially cultured in CD34⁺ HSPC medium at low density for 3 days before being electroporated with Cas9 RNP complexes. After a 3-day recovery phase, pDC differentiation was initiated. b) Expansion of HSPC during differentiation into HSPC-pDCs. c) Indel frequencies quantified by TIDE analysis for sgRNAs directed at MyD88, IFNAR1 and CCR5 (control) after the 3-day recovery phase. d) Numbers of HSPC-pDCs at day 21 for gene edited HSPC-pDCs. e) Western blot analysis showing knock-down of MyD88 in HSPC-pDCs. Indel frequencies at the respective targets are listed below the Western Blot. f-g) Surface expression levels (MFI) of CD123 (f) and CD304 (g) in primed and unprimed HSPC-pDCs with gene editing at MyD88, IFNAR1 and CCR5. h) Functional levels of type I IFN after stimulation with R837 (TLR7) or CpG-A (TLR9) in unprimed and primed HSPC-pDCs gene-edited at CCR5, MyD88 or CCR5. Data are ±SEM of five (b-d) or three (f) donors. Statistical analysis was performed using regular two-way ANOVA (a, c) followed by Bonferroni post hoc test.

FIG. 19. Genetic modifications are retained during cultivation and differentiation of HSPCs into HSPC-pDCs. a) Indel frequencies of HSPCs three days after electroporation with sgRNAs directed against MyD88, IFNAR1 and CCR5. b) Indel frequencies of the total population of cells or the enriched fraction of HSPC-pDCs at day 21 of culture for HSPCs electroporated with sgRNAs. Bars represent average Indel frequencies ±SEM of 5 donors.

### DETAILED DESCRIPTION

### Method for Producing pDCs

One aspect of the present invention provides a method for producing plasmacytoid dendritic cells (pDCs), said method comprising
providing hematopoietic stem and progenitor cells (HSPCs)
incubating said HSPCs in a first medium comprising cytokines and growth factors whereby said HSPCs are differentiated into precursor-pDCs
adding interferons (IFNs) to said first medium to obtain a second medium
   whereby said precursor-pDCs are differentiated into pDCs

Another aspect relates to a method for producing a plasmacytoid dendritic cells (pDCs), said method comprising
providing hematopoietic stem progenitor cells (HSPCs)
incubating said HSPCs in a first medium comprising cytokines and growth factor whereby said HSPCs are differentiated into precursor-pDCs
adding SCF and SR1 in a first medium to obtain high yield of pre-cursor pDCs
providing a second medium comprising interferons (IFNs)
adding said second medium to said first medium comprising pre-cursor pDCs, whereby said precursor-pDCs are transformed into activated and differentiated pDCs

“Hematopoietic stem cells” (HSCs) as used herein are multipotent stem cells that are capable of giving rise to all blood cell types including myeloid lineages and lymphoid lineages. Myeloid lineages may for example include monocytes and macrophages, neutrophils, basophils, eosinophils, erythrocytes, megakaryocytes/platelets and dendritic cells, whereas lymphoid lineages may include T-cells, B-cells and NK-cells.

In a preferred embodiment HSCs are Hematopoietic stem and progenitor cells (HSPCs)

HSCs or HSPCs are found in the bone marrow of humans, such as in the pelvis, femur, and sternum. They are also found in umbilical cord blood and in peripheral blood.

Stem and progenitor cells can be taken from the pelvis, at the iliac crest, using a needle and syringe. The cells can be removed as liquid for example to perform a smear to look at the cell morphology or they can be removed via a core biopsy for example to maintain the architecture or relationship of the cells to each other and to the bone.

The HSCs or HSPCs may also be harvested from peripheral blood. To harvest HSCs or HSPCs from the circulating peripheral blood, blood donors can be injected with a cytokine that induces cells to leave the bone marrow and circulate in the blood vessels. The cytokine may for example be selected from the group consisting of granulocyte-colony stimulating factor (G-CSF), GM-CSF granulocyte-macrophage colony-stimulating factor (GM-CSF) and cyclophosphamide. They are usually given as an injection into the fatty tissue under the skin every day for about 4-6 days.

The HSCs or HSPCs may also be harvested or purified from bone marrow. Stem cells are 10-100 times more concentrated in bone marrow than in peripheral blood. The hip (pelvic) bone contains the largest amount of active marrow in the body and large numbers of stem cells. Harvesting stem cells from the bone marrow is usually done in the operating room.

In another embodiment the HSCs or HSPCs are purified from human umbilical cord blood (UCB). In this embodiment, blood is collected from the umbilical cord shortly after a baby is born. The volume of stem cells collected per donation is quite small, so these cells are usually used for children or small adults.

The method is in certain embodiments an in vitro method.

Precursor-pDCs as used herein are generated after incubation of HSCs or HSPCs in the first medium, whereby HSCs or HSPCs differentiate into precursor-pDCs.

Plasmacytoid dendritic cells (pDCs) as used herein are generated after interferon priming and display a phenotype strongly resembling blood pDCs as described elsewhere herein.

In one preferred embodiment, the HSCs, such as HSPCs, are genetically modified. The HSCs or HSPCs preferably are genetically modified prior to pDC differentiation, i.e. prior to their incubation in the first medium comprising cytokines and growth factor.

The HSCs, such as HSPCs, may be modified by any methodology available in the art. In a preferred approach, the cells are genetically modified using delivery of single guide RNA (sgRNA) and/or adeno-associated virus using electroporation and/or transduction.

Preferred genetic modifications are knock-out of one or more co-stimulatory markers. Thus, in a preferred embodiment, the HSCs, such as HSPCs, have been genetically modified by knock-out of one or more of the co-stimulatory factors selected from the group consisting of IDO, CD85g and PD-L1.

### First Medium

The first medium is a differentiation medium, wherein HSCs are differentiated into precursor-pDCs. Thus, the first medium comprises differentiation factors.

Before differentiation of HSCs into precursor-pDCs, the HSCs may be cultured in a culture medium not comprising differentiation factors. The culture medium may be supplemented with conventional cell culture components such as serum, such as for example fetal calf serum, b-mercaptoethanol, antibiotics, such as penicillin and/or streptomycin, nutrients, and/or nonessential amino acids. Conventional cell culture components can also be substituted for conventional serum-free medium supplemented with conventional penicillin and/or streptomycin.

To initiate differentiation of HSCs into precursor-pDCs, differentiation factors, such as Flt3 ligand, thrombopoietin and/or at least one interleukin selected from interleukin-3, IFN-b and PGE2 are added to the medium. SCF and/or SR1 can also be used.

Thus, in a preferred embodiment said first medium comprises Flt3 ligand, thrombopoietin and/or at least one interleukin selected from interleukin-3, IFN-b and PGE2. More preferably, said first medium comprises Flt3 ligand, thrombopoietin and/or interleukin-3. In another preferred embodiment, the first medium comprises SCF and/or SR1

The concentration of thrombopoietin may for example be in the range of from 10 to 250 ng/mL, such as in the range of from 20 to 200 ng/mL, such as for example in the range of from 25 to 150 ng/mL, such as in the range of from 20 to 100 ng/mL or such as in the range of from 30 to 80 ng/mL. In one embodiment the concentration of thrombopoietin is 50 ng/mL.

The concentration of Flt3 ligand may for example be in the range of from 20 to 500 ng/mL, such as in the range of from 40 to 400 ng/mL, such as for example in the range of from 50 to 300 ng/mL, such as in the range of from 60 to 200 ng/mL or such as in the range of from 75 to 150 ng/mL. In one embodiment the concentration of Flt3 ligand is 100 ng/mL.

The concentration of interleukin-3 may for example be in the range of from 5 to 100 ng/mL, such as in the range of from 10 to 80 ng/mL, such as for example in the range of from 10 to 60 ng/mL, such as in the range of from 15 to 50 ng/mL or such as in the range of from 15 to 40 ng/mL. In one embodiment the concentration of interleukin-3 is 20 ng/mL.

In one preferred embodiment said first medium further comprises at least one cytokine selected from stem cell factor, interleukin-7 and StemRegenin 1. More preferably, said first medium comprises stem cell factor and StemRegenin 1.

The concentration of stem cell factor may for example be in the range of from 20 to 500 ng/mL, such as in the range of from 40 to 400 ng/mL, such as for example in the range of from 50 to 300 ng/mL, such as in the range of from 60 to 200 ng/mL or such as in the range of from 75 to 150 ng/mL. In one embodiment the concentration of stem cell factor is 100 ng/mL.

The concentration of StemRegenin 1 may for example be in the range of from 0.1 to 5 μM, such as in the range of from 0.5 to 3 μM, such as for example in the range of from 0.5 to 2 μM, such as in the range of from 0.5 to 1 μM, such as in the range of from 0.8 to 3 μM or such as in the range of from 1 to 4 μM. In one particular embodiment the concentration of stem cell factor is 1 μM.

In another preferred embodiment the first medium further comprises UM171. UM171 is a pyrimidoindole derivative that improves the expansion of primitive hematopoietic cells

The concentration of UM171 for example be in the range of from 10 to 500 nM, such as in the range of from 10 to 300 nM, such as for example in the range of from 10 to 250 nM, such as in the range of from 15 to 50 nM, such as in the range of from 20 to 200 nM or such as in the range of from 40 to 150 nM.

The HSPCs are incubated in the first medium under conditions that are typical for human cell cultures and well known to the skilled person. Typical conditions for incubation of cell cultures are for example a temperature of 37° C., 95% humidity and 5% CO₂.

In one embodiment the HSPCs are incubated for at least 1 day, such as at least 2 days, at least 3 days, such as for example at least 4 days, such as at least 5 days, at least 6 days, such as for example at least 7 days, such as at least 8 days, at least 9 days, such as for example at least 10 days, such as at least 12 days, at least 14 days in said first medium. In a more preferred embodiment the culture is incubated for at least 16 days, such as at least 18 days, at least 20 days or such as for example at least 21 days in said first medium.

The HSCs may for example be incubated for 1 week, 2 weeks, 3 weeks or 4 weeks in said first medium. In a preferred embodiment said HSPCs are incubated for 21 days in said first medium.

In one embodiment the first medium is refreshed during the incubation period. The medium may for example be refreshed every second day, every third day or every fourth day during the incubation period. The first medium is preferably refreshed with medium containing one or more components of the first medium as described herein and above. Preferably the medium is refreshed with medium comprising the cytokines.

Incubation of said HSCs in said first medium may lead to an average yield of 60-100 precursor-pDCs per HPC.

### Second Medium

After incubation of HSPCs in the first medium, wherein HSCs are differentiated into precursor-pDCs, IFNs are added to the first medium thereby obtaining a second medium.

Alternatively, a second medium is provided, which comprises IFNs, such as IFN type I, IFN type II and/or IFN type III.

The inventors of the present invention have found that the presence of IFNs in the medium increases the viability of the pDCs and also induces phenotypic and functional characteristics, which are similar to blood pDCs. Thus, IFN treatment can prime precursor-pDCs to adopt a blood pDC phenotype. The immature precursor-pDCs thereby develop into a more mature phenotype.

In one embodiment said second medium comprises IFN-α, IFN-γ and/or IFN-β. In one preferred embodiment said second medium comprises IFN-γ and/or IFN-β. Preferably, said second medium comprises IFN-γ and IFN-β.

In another preferred embodiment said second medium comprises interleukin-3 (IL-3). In the embodiment, wherein the first medium comprises IL-3, IL-3 may be added to the medium again, for example together with the interferons. It is understood that the three components can be added in any order. In a particular preferred embodiment said second medium comprises IFN-γ, IFN-β and IL-3.

In one embodiment the concentration of IFN-γ is in the range of from 5 to 400 U/mL.

The concentration of IFN-γ may for example be in the range of from 30 to 400 U/mL, such as in the range of from 30 to 300 U/mL, or more preferably in the range of from 30 to 250 U/mL.

In another embodiment the concentration of IFN-γ may is in the range of from 50 to 400 U/mL, such as in the range of from 100 to 300 U/mL, or more preferably in the range of from 100 to 250 U/mL.

The concentration of IFN-γ may for example be in the range of from 5 to 200 U/mL, such as in the range of from 5 to 100 U/mL, such as in the range of from 10 to 80 U/mL, such as in the range of from 15 to 60 U/mL, or more preferably in the range of from 20 to 40 U/mL.

The concentration of IFN-γ may for example be at least 30 U/mL.

In one embodiment the concentration of IFN-β is in the range of from 5 to 400 U/mL.

The concentration of IFN-β may for example be in the range of from 30 to 400 U/mL, such as in the range of from 30 to 300 U/mL, or more preferably in the range of from 30 to 250 U/mL.

In another embodiment the concentration of IFN-β may is in the range of from 50 to 400 U/mL, such as in the range of from 100 to 300 U/mL, or more preferably in the range of from 100 to 250 U/mL.

In one preferred embodiment the concentration of IFN-β is 250 U/mL.

The concentration of IFN-β may for example be in the range of from 5 to 200 U/mL, such as in the range of from 5 to 100 U/mL, such as in the range of from 10 to 80 U/mL, such as in the range of from 15 to 60 U/mL, or more preferably in the range of from 20 to 40 U/mL.

The concentration of IFN-β may for example be at least 30 U/mL.

The precursor-pDCs are incubated in the second medium under conditions that are typical for human cell cultures and well known to the skilled person. Typical conditions for incubation of cell cultures are for example a temperature of 37° C., 95% humidity and 5% CO₂.

In one embodiment said precursor-pDCs are incubated in said second medium for at least 1 hour, such as at least 5 hours, such as for example at least 10 hours, such as at least 15 hours or such as at least 20 hours in said second medium. In one preferred embodiment precursor-pDCs are incubated for at least 24 hours in said second medium.

In another embodiment said precursor-pDCs are incubated in said second medium for at least 1 day, at least two days, at least three days or at least 4 days.

In yet another embodiment, said precursor-pDCs are incubated in said second medium for 1 to 72 hours, such as from 5 to 72 hours, such as for example from 10 to 72 hours, such as from 20 to 72 hours, such as for example from 24 to 72 hours or such as from 48 to 72 hours.

In another embodiment said precursor-pDCs are incubated in said second medium for 1 to 7 days, such as from 1 to 6 days, such as for example from 1 to 5 days, such as from 1 to 4 days, such as for example from 1 to 3 days or for example from 1 to 2 days.

### pDC Phenotype

In one preferred embodiment of the present invention, the pDCs express TNF-related apoptosis-inducing ligand (TRAIL). TRAIL, which is also designated CD253, is a ligand involved in killing cancer cells by interacting with TRAIL receptors (death receptor 5, DR5) on the surface of cancer cells and thereby triggering apoptosis.

The pDCs obtained by incubating in the second medium are matured cells having a surface phenotype that strongly resembles blood pDCs. In a preferred embodiment, said pDCs express CD123, CD303, CD304, CD4 and/or HLA-DR.

In another preferred embodiment said pDCs express IFN type I, IFN type II, IFN type III and/or proinflammatory cytokines.

The pDC may in one preferred embodiment express Toll-like receptors, such as for example Toll-like receptor 7 (TLR7) and/or Toll-like receptor 9 (TLR9).

In yet another preferred embodiment said pDCs express Interferon regulatory factor 7 (IRF7).

In another preferred embodiment said pDCs express Cluster of differentiation 80 (CD80), which is a protein found on Dendritic cells, activated B cells and monocytes that provides a costimulatory signal necessary for T cell activation and survival.

The pDC may in a preferred embodiment also express proteins characteristic for antigen presenting cells such as for example Cluster of Differentiation 86 (CD86) and/or Cluster of Differentiation 40 (CD40). CD86 is a protein expressed on antigen-presenting cells that provides costimulatory signals necessary for T cell activation and survival, whereas CD40 is a costimulatory protein found on antigen presenting cells and is required for their activation.

Preferably, said pDCs express CD40, CD80, CD83 and/or CD86. In another preferred embodiment said pDCs express interleukin 6 (IL-6).

The pDCs may express any combination of the proteins mentioned above.

The pDCs may be further enriched using immunomagnetic separation methods that are based on the attachment of small magnetizable particles to cells via antibodies or lectins. When the mixed population of cells is placed in a magnetic field, those cells that have beads attached will be attracted to the magnet and may thus be separated from the unlabelled cells. For example, antibodies coating paramagnetic beads can bind to antigens present on the surface of cells thus capturing the cells and facilitate the concentration of the bead-attached cells. The concentration process can be created by a magnet placed on the side of the test tube bringing the beads to it.

Cells can also be separated using a “negative sorting” method, wherein unwanted cell types are immunomagnetically labelled. This process may require a cocktail of antibodies. The labelling procedure is the same as for positive sorting except that the unlabelled fraction of the cell population is retained and the labelled cells are discarded.

Thus, in one embodiment the method as described herein above, further comprises a step of immunomagnetic negative selection to enrich for differentiated pDCs.

### Genetically Modified HSPC-pDCs

In one aspect, the pDCs provided by the methods disclosed herein are genetically modified. Thus, one aspect of the present invention relates to genetically modified pDCs.

The HSCs, such as HSPCs, which are used for production of pDC in the presently disclosed method may thus be genetically modified. The HSCs or HSPCs preferably are genetically modified prior to pDC differentiation, i.e. prior to their incubation in the first medium comprising cytokines and growth factor.

The HSCs, such as HSPCs, may be modified by any methodology available in the art. In a preferred approach, the cells are genetically modified using delivery of single guide RNA (sgRNA) and/or adeno-associated virus using electroporation and/or transduction.

Preferred genetic modifications are knock-out of one or more co-stimulatory markers. Thus, in a preferred embodiment, the HSCs, such as HSPCs, have been genetically modified by knock-out of one or more of the co-stimulatory factors selected from the group consisting of IDO, CD85g and PD-L1.

Thus, in one aspect of the present disclosure, genetically modified pDCs are provided, which have been modified in order to make them more suitable for therapeutic use, e.g. for use as vaccines. Embodiments of such genetically modified pDCs include pDCs, wherein one or more co-stimulatory factors have been knocked out, such as co-stimulatory factors selected from the group consisting of IDO, CD85g and PD-L1.

Such genetically modified pDCs can be generated by
providing hematopoietic stem progenitor cells (HSPCs)
genetically modifying said HSPCs, i.e. by knock-out of a co-stimulatory factors selected from the group consisting of IDO, CD85g and PD-L1
incubating said HSPCs in a first medium comprising cytokines and growth factor whereby said HSPCs are differentiated into precursor-pDCs
adding SCF and SR1 in a first medium to obtain high yield of pre-cursor pDCs
providing a second medium comprising interferons (IFNs)
adding said second medium to said first medium comprising pre-cursor pDCs, whereby said precursor-pDCs are transformed into activated and differentiated, genetically modified pDCs.
   The disclosure also in one aspect relates to genetically modified pDCs obtained or obtainable be aforementioned method.

### Antigen Presenting Cells

Since dendritic cells (DC) are potent antigen presenting cells (APC), they are considered as a powerful tool to deliver the signals essential for the activation of immune system. When a dendritic cell recognizes a pathogen-associated molecular pattern, antigen is phagocytosed and the dendritic cell becomes activated, upregulating the expression of MHC class II molecules. It also upregulates several co-stimulatory molecules that can interact with CD28 on the surface of a CD4+ T cell and induce a T cell response.

The inventors have found that the pDCs provided from the method described herein, are capable of taking up antigen and presenting the antigen on its cellular surface, cf. example 2. In addition, the pCDs are amenable to genetic modification, which improves their therapeutic capacity; cf. examples 3 and 4.

Accordingly, the method as described above may further comprise a step of incubating said pDCs with at least one antigen or neoantigen. Incubation of pDCs with antigens or neoantigens results in pDCs being loaded with antigens/neoantigens and the formation of antigen presenting cells (APCs). In one embodiment the antigen is a cancer specific antigen or neoantigen. In another embodiment said antigen is a pathogen specific antigen. In yet another embodiment said antigen is a virus-, bacteria- or parasite specific antigen.

Thus, an embodiment of the present invention relates to a method for producing antigen/neoantigens presenting cells (APCs), said method comprising
providing hematopoietic stem and progenitor cells (HSPCs)
exposing said HSPCs to a first medium comprising cytokines and growth factor to induce differentiation of HSPCs into precursor-pDCs
culturing said precursor-pDCs in a second medium comprising interferons to induce differentiation of precursor-pDCs into mature pDCs
incubating pDCs with at least one antigen/neoantigens
   whereby APCs are generated.

One embodiment relates to a method for producing antigen/neoantigens presenting cells (APCs), said method comprising
providing hematopoietic stem progenitor cells (HSPCs)
incubating said HSPCs in a first medium comprising cytokines and growth factor whereby said HSPCs are differentiated into precursor-pDCs
adding SCF and SR1 in a first medium to obtain high yield of pre-cursor pDCs
providing a second medium comprising interferons (IFNs)
adding said second medium to said first medium comprising pre-cursor pDCs, whereby said precursor-pDCs are transformed into activated and differentiated pDCs, and
incubating said pDCs with at least one antigen/neoantigens
   whereby APCs are generated.

The HSPCs, precursor-pDCs and pDCs are incubated under conditions that are typical for human cell cultures and well known to the skilled person. Typical conditions for incubation of cell cultures are for example a temperature of 37° C., 95% humidity and 5% CO₂.

As used herein, APCs are pDCs that have been loaded with anitigens. The APCs can display the antigen complexed with major histocompatibility complexes (MHCs) on their surfaces; this process is known as antigen presentation. T cells may recognize these complexes using their T cell receptors (TCRs). These cells process antigens and present them to T-cells. Preferably, the APC can present the antigen to both helper and cytotoxic T cells. The APC may also be capable of cross-presentation, whereby they present exogenous antigen on MHC class I molecules to cytotoxic T cells.

Preferably, the APCs express MHC class II molecules on their cell surface. The T cell recognizes and interacts with the antigen-class II MHC molecule complex on the surface of the APC, leading to activation of the T cell.

Helper T cells can recognize exogenous antigen presented on MHC class II, whereas cytotoxic T cells can recognize endogenous antigen presented on MHC class I. Thus, in one embodiment the APCs can induce a CD4+ helper T cell response. In another embodiment the APCs can induce a CD8+ cytotoxic T cell response. Preferably, APCs of the present invention can induce a CD4+ helper T cell response and a CD8+ cytotoxic T cell response.

As used herein, the term “antigen” defines a molecule recognized by cells of the immune system and capable of triggering an immune reaction. The antigen may be any protein, peptide or fragment thereof against which it is desirable to raise an immune response.

The antigens of the present invention may be natural or modified, tumor, bacterial or viral antigens, such as peptides, proteins, glycopeptides, glycoproteins or phosphorylated proteins.

For example, the antigens are peptides which can be obtained from antigenic proteins of tumour or viral origin.

Thus, in one embodiment said antigen is a tumour-specific antigen. The term “tumour-specific” antigen encompasses any cancerous, transformed or malignant cell as well as antigens from solid tumours.

In another embodiment said antigen is a pathogen specific antigen. In another embodiment said antigen is a virus-, bacteria- or parasite specific antigen.

Thus, the APCs of the present invention may prime the immune system to attack malignant cells, such as cells infected with a virus or a pathogen or in particular cancer cells.

### Cell Populations

The present invention also provides a pDC population and/or an APC population obtained by the method according to any of claims the preceding claims.

Embodiments described herein referring to pDCs may also refer to a pDC population. Similarly, embodiments described herein referring to APCs may also refer to an APC population.

Thus, the pDC population and/or the APC population expresses the proteins as defined herein above for pDCs obtained by the method of the present invention. That is, the pDC population and/or APC population has the same phenotypic characteristic as described for the pDCs and/or APCs obtained by the method of the present invention.

Thus, in a preferred embodiment the pDC population and/or the APC population expresses TRAIL.

In one embodiment said pDC population and/or the APC population expresses CD123, CD303, CD304, CD4 and/or HLA-DR. In another embodiment said pDCs line expresses IFN type I, IFN type II, IFN type III and/or proinflammatory cytokines. In yet another embodiment said pDCs line and/or the APC population expresses IRF7, TLR7 and/or TLR9. In a further embodiment, said pDCs population and/or said APC line expresses CD40, CD80, CD83 and/or CD86. Said pDCs population and/or said APC line may in one embodiment express IL-6.

The pDC line and/or said APC line may express any combination of the proteins mentioned above.

### Vaccines

Induction of T cell responses is dependent on interactions with antigen-presenting cells (APCs), in particular dendritic cells (DCs), which present tumor-specific antigens, such as neoantigens as discussed below. Thus, to induce T cell responses, cancer patients can be vaccinated with a vaccine comprising APCs

As shown elsewhere herein, pDCs produced according to the methods provided herein are capable of presenting specific antigens and/or are amenable to genetic modification. Thus, the pDCs can be used for induction of T cell responses, which makes them suitable for use as vaccines, in particular cancer vaccines. Thereby, a personalized antigen vaccine is obtained that specifically targets the identified tumor antigens. In cancer, certain so-called tumor neoantigens may arise due to one or more mutations in the tumor genome leading to a change in the amino acid sequence of the protein in question. Since these mutations are not present in normal tissue, the side-effects of the treatment directed towards the tumor associated antigens do not arise with an immunologic treatment towards tumor neoantigens. The average number of somatic, tumor-specific non-synonymous mutations is for malignant melanoma between 100 and 120. Some of the genetic alterations can be recognized by the immune system, representing ideal antigens. Animal models have confirmed the utility of immunization with tumor neoantigens

The term “tumor neoantigen” is used for any tumor specific antigen comprising one or more mutations as compared to the host's exome and is used synonymously with the term cancer neoantigen. The term “tumor neoepitope is used for any immunogenic mutation in a tumor antigen and is used synonymously with the term cancer neoepitope.

The present invention provides in one aspect, a vaccine comprising an immunologically effective amount of the APCs as defined herein above. The APCs are produced by incubating pDCs provided in the method disclosed herein with at least one antigen, which is preferably a cancer-specific antigen/neoantigen or an antigen specific to an infectious microorganism.

By “immunologically effective amount” is meant an amount that is sufficient to induce an immune response.

In particularly, the vaccine designed for evoking a cell-mediated immune response through activation of T cells against the specific antigens presented on the APCs. T cells recognize epitopes/neoepitopes, when they have been processed and presented complexed to a MHC molecule as discussed below.

Generally, vaccines are used to prevent or reduce the severity of a physiological or clinical condition, such as an infectious disease or cancer. For example, the vaccine may be used for preventing cancer in a patient, or reducing or destroying tumor cells already present in a patient.

The vaccine can be administered in a therapeutically effective amount or in a prophylactically effective amount. Therapeutically and prophylactically effective amounts are defined herein above.

### Pharmaceutical Formulation

Whilst it is possible for the APCs or pDCs or vaccines of the present invention to be administered alone, it is preferred to present them in the form of a pharmaceutical formulation.

Accordingly, the present invention further provides a pharmaceutical formulation, which comprises pDCs and/or APCs and/or vaccines of the present invention and a pharmaceutically acceptable carrier.

Suitable carriers and the formulation of such pharmaceuticals are known to a person skilled in the art.

The pharmaceutical formulation of the present invention may be formulated for parenteral administration and may be presented in unit dose form in ampoules, pre-filled syringes, small volume infusion or in multi-dose containers. The formulations may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain agents such as preserving, wetting, emulsifying or suspending, stabilizing and/or dispersing agents. Other examples of carriers include phosphate-buffered common salt solutions, water, wetting agents and/or sterile solutions. The dosage requirements will vary with the particular composition employed, the route of administration and the particular individual being treated.

Thus, a suitable dose or a therapeutically effective dose level for a particular subject or patient will depend upon a variety of factors such as the disorder being treated and the severity of the disorder and the patient's age, weight, health and sex

The suitable is preferably determined by the attending physician. The dosage can be adjusted by the individual physician in the event of any contraindications. The dosage can vary, and can be administered in one or more doses daily, for one or several days.

Ideally, the patient or subject to be treated by the present formulation will receive a pharmaceutically or therapeutically effective amount of the formulation or the cells in the maximum tolerated dose, generally no higher than that required before drug resistance develops.

Thus, it is preferred that the pharmaceutical formulation is administered in a therapeutically effective amount.

By “therapeutically effective amount” herein is meant a dose that produces the therapeutic effects for which it is administered. The exact dose will depend on the clinical condition or disorder to be treated, and can be ascertained by one skilled in the art using known techniques.

In further embodiments, the pharmaceutical formulation can be administered in a prophylactically effective amount. A prophylactically effective amount is an amount effective for prevention of a disease or condition.

In one embodiment the pharmaceutical composition comprises at least one adjuvant.

The adjuvant is preferably an immunostimulatory agent that may include agents capable of activating dendritic cells and stimulating their ability to promote T cell activation.

In one embodiment the adjuvant is an agonist for CD40 such as for example soluble CD40 ligand, or an agonist antibody specific for CD40 or an antagonist of CD40 such as an anti-CD40 antibody.

In another embodiment the adjuvant is an agonist of CD28, CD27 or OX40, a CTLA-4 antagonist.

The adjuvant may for example be a Toll-like receptor (TLR) agonist, which activates a Toll-like receptor.

### Administration

The pharmaceutical formulation of the present invention may be administered by parenteral administration.

Parenteral administration is any administration route not being the oral/enteral route whereby the medicament avoids first-pass degradation in the liver. Accordingly, parenteral administration includes any injections and infusions, for example bolus injection or continuous infusion, such as intravenous administration, intramuscular administration, subcutaneous administration.

In a preferred embodiment, the pharmaceutical formulation is administered by injection.

In one embodiment the pharmaceutical formulation of the present invention may be injected into the site of action, for example into the diseased tissue or to an end artery leading directly to the diseased tissue.

In particular, the pharmaceutical formulation may be injected intra-muscularly, directly in the lymph nodes or into tumorigenic tissue.

### Therapeutic Methods

A further aspect of the present invention relates to pDCs and/or APCs as defined herein above for use in the treating, preventing and/or ameliorating an infectious disease or cancer. Other aspects relate to the pharmaceutical formulation and/or a pharmaceutical formulation as defined herein above use in the treatment of an infectious disease or cancer.

One aspect also relates to a method of treating, preventing and/or ameliorating an infectious disease and/or cancer, said method comprising administering a therapeutically efficient amount of pDCs and/or APCs to a subject in need thereof. Other methods are also provided of treating, preventing and/or ameliorating an infectious disease and/or cancer, said method comprising administering a therapeutically efficient amount of a pharmaceutical formulation and/or a vaccine as defined herein to a subject in need thereof.

The infectious disease may for example be a bacterial infection or a viral infection. The viral infection may for example be due to influenza virus, human immunodeficiency virus (HIV), hepatitis B virus (HBV), hepatitis C virus (HCV), Zika virus, HPV or Herpes virus.

The cancer may for example be melanoma, breast cancer, ovarian cancer, head-and-neck cancer, brain cancer, AML, bladder cancer, Carcinoma of Unknown Primary, Cervical Cancer, Colorectal Cancer, liver cancer, Hodgkin Lymphoma, Metastatic Cancer, Pancreatic Cancer, lung cancer, prostate cancer or virus-induced cancers. In a preferred embodiment said cancer is TRAIL-specific cancer.

The term “TRAIL-specific cancer” as used herein refers to cancer, wherein the cancer cells express the TRAIL receptor, including TRAIL-R1 and TRAIL-R2. The TRAIL receptor is expressed on the surface of the cancer cells. The presence of TRAIL receptor can for example be detected using immunologic assays, such as ELISA or Flow Cytometry, and other generally well-known assays within molecular biology, such as PCR, qPCR or next-generation sequencing.

Preferably said subject is an individual, such as a human.

A therapeutically efficient amount is defined herein above.

In one embodiment said infectious disease is a bacterial infection or a viral infection. In a preferred embodiment said cancer is TRAIL-specific cancer.

Preferably, the method comprises administering a therapeutically effective amount of a pharmaceutical composition as defined herein above.

Administration forms are as described herein above.

### Kit

The present invention also provides a kit comprising a pDC cell line as defined herein above, and elements associated with immunological use. In one embodiment the kit further comprises at least one antigen.

The present invention also provides a kit comprising an APC line as defined herein above, and elements associated with immunological use. In one embodiment the kit further comprises at least one antigen.

### EXAMPLES

### Materials and Methods

Ex Vivo Generation of pDCs From CD34⁺ HSPC

CD34⁺ HSPCs were purified from human umbilical cord blood (UCB). Briefly, mononucleated cells were recovered by standard Ficoll-Hypaque (GE Healthcare) density-gradient centrifugation. CD34⁺ cells were then isolated using anti-CD34 immunomagnetic beads (positive selection) following the manufacturer's instructions (EasySep™ Human cord blood CD34⁺ positive selection kit, STEMCELL Technologies). CD34⁺ HSPC were either freshly used or cryo-preserved until future use. To obtain differentiation of pDCs, CD34⁺ HSPC were plated in 48-well plates in RPMI-1640 medium (Lonza), supplemented with 10% heat inactivated FCS (HyClone®), 600 μg/mL L-glutamine (Sigma), 200 U/mL penicillin and 100 μg/mL streptomycin (Gibco®, Life Technologies). Furthermore, the cytokines Flt3-L, TPO, and IL-3 were added as a baseline. In addition to the baseline, the cytokines SCF, IL-7 and StemRegenin 1 were tested individually or in combination at various concentrations. Cells were cultured at 37° C., 95% humidity, and 5% CO₂ for 21 days. Medium was refreshed every 4^{th} day with medium containing the designated cytokines. Total cell numbers during the cultivation period were determined using the cell counter Moxi Z™ (ORFLO Technologies). At day 21 HSPC-pDCs were enriched using a negative selection kit that depletes non-pDCs, according to the manufacturer's protocol (EasySep™ Human Plasmacytoid DC Enrichment kit, STEMCELL Technologies).

### pDC Enrichment From Peripheral Blood

Peripheral blood mononuclear cells (PBMC) were prepared by Ficoll-Hypaque density centrifugation of normal healthy blood donor buffy coats. Blood pDCs were enriched by negative selection following the instructions by manufacturer (EasySep™ Human Plasmacytoid DC Enrichment kit, STEMCELL Technologies).

### Priming of HSPC-pDCs

HSPC-pDCs were cultured in RPMI-1640 medium with 10% heat inactivated FCS, 600 μg/mL L-glutamine, 200 U/mL penicillin and 100 μg/mL streptomycin, but depleted for growth factors used for the cultivation process. As described in individual figure legends, medium was supplemented with IFN-β (PBL Assay Science), IFN-γ (PeproTech) or both cytokines simultaneously (IFN-β+γ). After 24 (day 21+1) or 72 (day 21+3) hours, cells were washed in warm medium, and phenotypically or functionally characterized.

### Cell Stimulation

For functional characterization, cells were seeded in 96-well plates. Cells were left untreated or stimulated with stimuli as shown in table 1. During stimulation cells were kept in medium with 20 ng/mL IL-3. Twenty hours post stimulation supernatants were harvested and kept at −20° C. until cytokine quantifications. For phenotypic characterization, primed cells were seeded out in 48-well plates and stimulated for 24 hours before phenotypic analysis by flow cytometry.

TABLE 1

| | | | Targeted | |
|---|---|---|---|---|
| Agonist | Manufacturer | Cat. # | PRR | Concentration |
| R837 - imiquimod | InvivoGen | tlrl-imq | TLR7 | 2.5 μg/mL |
| R848 - resiquimod | InvivoGen | tlrl-r848 | TLR7, | 0.5 μg/mL |
| | | | TLR8 | |
| CpG ODN 2216 | InvivoGen | tlrl-2216 | TLR9 | 12 μg/mL |
| (Class-A) | | | | |
| CpG ODN 2006 | InvivoGen | tlrl-bw006 | TLR9 | 12 μg/mL |
| (Class-B) | | | | |

### Measurement of Functional Type I IFN

Bioactive functional type I IFN was quantified in supernatants using the reporter cell line HEK-Blue™ IFN-α/β (Invivogen). The cell line was maintained in DMEM+GlutaMax™-I (Gibco®, Life Technologies), supplemented with 10% heat-inactivated FCS, 100 μg/mL streptomycin and 200 U/mL penicillin, 100 μg/mL normocin (InvivoGen), 30 μg/mL blasticidin (InvivoGen) and 100 μg/mL zeocin (InvivoGen). Cells were passaged using 1× trypsin (Gibco®, Life Technologies). To ensure optimal type I IFN response and stable expression of the plasmids encoding hIRF9, hSTAT2 and SEAP, cells were not passaged more than 20 times. For measurement of functional type IFN cells were seeded at 3×10⁴ cells/well in 96-well plates in 150 μL medium. Cells were grown as previously described, but without blasticidin+zeocin. The following day, 50 μL of supernatant from stimulated cells or a type I IFN standard range was added to the cells. After 24 hours of incubation, 20 μL of supernatant from the HEK-Blue cells were subsequently added to 180 μL QUANTI-Blue™. SEAP activity was assessed by measuring optical density (OD) at 620 nm on a microplate reader (ELx808, BioTEK). The standard range was made with IFN-α (IFNa2 PBL Assay Science) and ranged from 2 to 500 U/mL.

### Enzyme-Linked Immunosorbent Assay (ELISA)

Protein levels of IL-6 in supernatants were evaluated using ELISA kits (Biolegend) following the manufacturers instructions.

### Morphology of Cells

To examine the morphology of cells at day 21, cells were smeared out on glass slides and fixed with methanol. May-Grünwald Giemsa Staining (Merck) was performed according to the manufacturers protocol. Slides were examined using a Leica DFC295 microscope.

### Phenotypic Analysis With Flow Cytometry

Immunophenotypical analysis was performed using flow cytometry. Briefly, 2×10⁵ cells were spun down and resuspended in PBS with 0.5% BSA and 0.09% NaN₃. Cells were blocked on ice for 30 min before being stained with fluorochrome-conjugated antibodies for 30 min. After three washing steps, cells were fixed in 0.9% formaldehyde. Fluorescence intensities were measured on a LSR Fortessa flow cytometer (Becton Dickinson) with four lasers (405, 488, 561 and 640 nm) and 18 photomultiplier (PMT) detectors. Data were collected using the FACSDiva software (Becton Dickinson). For some experiments data were collected using the NovoCyte Analyzer (ACEA Biosciences, Inc) equipped with three lasers (405, 488 and 640 nm) and 13 PMT detectors and data collected using NovoExpress Flow. Antibodies used to determine phenotype of cells are listed in Table 2. Data were analyzed using FlowJo software (version 10, Tree Star, Ashland, Oreg., USA). Individual gating strategies are outlined in figure legends. FMO controls were utilized to distinguish populations.

| Antibodies/ | | | | |
|---|---|---|---|---|
| viability dye | Fluorochrome | Supplier | Clone | Panel |
| Linage cocktail 1 | FITC | BD | CD3: SK7, | 1 |
| (CD3, CD14, | | | CD14: MϕP9, | |
| CD16, CD19, | | | CD16: 3G8, | |
| CD20, CD56) | | | CD19: SJ25C1, | |
| | | | C20: L27, | |
| | | | CD56: | |
| | | | NCAM16.2 | |
| CD11c | APC | BD | B-ly6 | 1 |
| CD123 | PE | eBioscience | 6H6 | 1, 2 |
| CD303a | PE-Cy7 | eBioscience | 201A | 1 |
| CD304 | BV421 | Biolegend | 12C2 | 1, 2 |
| HLA-DR | BV650 | BD | G46-6 | 1 |
| CD4 | PerCP-Cy5.5 | BD | RPA-T4 | 1 |
| Viability (7-AAD) | 7-AAD | BD | | 1 |
| Zombie Aqua | BV510 | Biolegend | | 2 |
| Fixable Viability | | | | |
| dye | | | | |
| CD34 | PE | eBioscience | 4H11 | |
| CD40 | PE | BD | 5C3 | 3 |
| CD80 | FITC | Biolegend | 2D10 | 3 |
| CD83 | PE-Cy7 | BD | HB15e | 3 |
| CD86 | BV605 | BD | 2331 (FUN-1) | 3 |

### Viability Over Prolonged Culture

To assess the viability over prolonged culture of HSPC-pDCs or blood pDCs, cells were collected at each time point and subsequently analyzed using flow cytometry. Cells were stained with Zombie aqua as a viability dye, and viable cell counts were measured and normalized to a fixed number of counting beads (CytoCount beads, Dako).

### Quantitative Real-Time PCR

Gene expression levels of IFN-α2, IFN-α4, IFN-α16, IRF7, TLR7 and TLR9 were determined by real-time PCR, using TaqMan detection systems. Expression levels were normalized to DAG1, and data are presented as relative expression levels. The following PCR primers were used: IFN-α2; Hs00265051_s1, IFN-α4; Hs01681284_sH, IFN-α16; Hs03005057_sH, TLR7; Hs01933259_s1, TLR9; Hs00370913_s1, DAG1; Hs00189308_m1.

### Antigen Presentation

Enriched HSPC-pDCs were primed with IFN-β+γ for 72 hours and subsequently stimulated with R837 (2.5 μg/mL). After 24 hours stimulation, HSPC-pDCs were pulsed with 1 μg per peptide/mL of CMV (ProMix™ CMV Peptide Pool, Prolmmune) or left untreated. After three hours cells were washed and subsequently co-cultured with PBMCs from CMV seropositive donors. As controls, PBMCs from the same donors were stimulated with 1 μg per peptide/mL of CMV or left untreated. PBMCs and HSPC-pDCs were co-cultured at a cell ratio of 10:1 (10⁵ PBMC and 10⁴ HSPC-pDCs) in duplicates in 96-well plates, 200 μL 10% RPMI supplemented with 20 ng/mL IL-3. After 20 hours incubation supernatants were harvested and T-cell activation evaluated by measuring levels of IFN-γ.

### Screening of sgRNAs

sgRNAs were initially screened for the capacity to induce insertions or deletions (Indels) in the immortalized K562 cell line (ATCC). K565 (ATCC) were maintained in RPMI 1640 (Lonza) supplemented with 10% FCS (HyClone®), 100 μg/mL streptomycin and 200 U/mL penicillin, and 2 mM L-glutamine. Two annealed oligonucleotides carrying the sgRNA target sequences were cloned into pX330 (pX330-U6-Chimeric_BB-CBh-hSpCas9 was a gift from Feng Zhang, Addgene plasmid #42230). The pX330 plasmid contains a human U6 promotor driving the expression of the sgRNA and a SpCas9 expression cassette (for sgRNA sequences see Table 3 below).

| Name | Sequence | SEQ ID NO | Name | Sequence | SEQ ID NO |
|---|---|---|---|---|---|
| MyD88 | 5′- | 1 | IFNAR1 | 5′- | 9 |
| #1 | GTTCTTGAACGTGCGGAC | | #1 | GACCCTAGTGCTCGTCGC | |
| | AC-3′ | | | CG-3′ | |
| MyD88 | 5′- | 2 | IFNAR1 | 5′- | 10 |
| #2 | GCTGCTCTCAACATGCGA | | #2 | GGGCGCGACGACCCTAG | |
| | GTG-3′ | | | TGC-3′ | |
| MyD88 | 5′- | 3 | IFNAR1 | 5′- | 11 |
| #3 | ACTGGACCGCGCTGGCG | | #3 | GCTCGTCGCCGTGGCGC | |
| | GAGC-3′ | | | CAT-3′ | |
| MyD88 | 5′- | 4 | IFNAR1 | 5′- | 12 |
| #4 | GCTTGAACGTGCGGACAC | | #4 | TAGTGCTCGTCGCCGTGG | |
| | AGG-3′ | | | CGC-3′ | |
| MyD88 | 5′- | 5 | IFNAR1 | 5′- | 13 |
| #5 | CGCTGAGGCTCCAGGAC | | #5 | AGTGCTCGTCGCCGTGG | |
| | CGC-3′ | | | CGC-3′ | |
| MyD88 | 5′- | 6 | IFNAR1 | 5′- | 14 |
| #6 | CCTGTCTCTGTTCTTGAAC | | #6 | GTGCTCGTCGCCGTGGC | |
| | GC-3′ | | | GCCA-3′ | |
| MyD88 | 5′- | 7 | | | |
| #7 | CTGGCTGCTCTCAACATG | | | | |
| | CGC-3′ | | | | |
| MyD88 | 5′- | 8 | CCR5 | 5′- | 15 |
| #8 | GTGTCTCTGTTCTTGAAC | | (control) | GCAGCATAGTGAGCCCA | |
| | GTG-3′ | | | GAA-3′ | |

Delivery of sgRNA-px330 plasmid to cells was performed by nucleoporation using the Lonza 4D-Nucleofector™ System (program FF-123). Three days after transfection, Indel frequencies were quantified using TIDE (Tracking of Indels by Decomposition); Genomic DNA was extracted and PCR amplicons spanning the sgRNA target site were generated (for primers see Table 4, below).

| Primer | Sequence | SEQ ID NO | Primer | Sequence | SEQ ID NO |
|---|---|---|---|---|---|
| MyD88 | 5′- | 16 | IFNAR1 | 5′- | 18 |
| fwd | CTCCGTGGAAGAACTGT | | fwd | GGAGTCGTCCTGGAATGC- | |
| | GGC-3′ | | | 3′ | |
| MyD88 | 5′- | 17 | IFNAR1 | 5′- | 19 |
| rev | GGCGGCTGTATCCAAC | | rev | ACCTCGAGAACTGACAATT | |
| | GC-3′ | | | ATGC-3′ | |
| | | | CCR5 | 5′- | 20 |
| | | | fwd | GCACAGGGTGGAACAAGAT | |
| | | | | GG-3′ | |
| | | | CCR5 | 5′- | 21 |
| | | | rev | CACCACCCCAAAGGTGACC | |
| | | | | GT-3′ | |

Purified PCR products were then Sanger-sequenced and Indel frequencies quantified using the TIDE software (http://tide.nki.nl). A reference sequence (mock-transfected sample) was used as a control.

### Making Genetically Modified HSPC-pDCs

sgRNAs directed at MyD88, IFNAR1 and CCR5 were synthesized by Synthego or TriLink Technologies with the three terminal nucleotides in both ends chemically modified with 2′-O-methyl-3′-phosphorothioate [28]. Thawed CD34⁺ HSPC were initially pre-cultured at low density (10⁵ cells/mL) for 3 days in CD34⁺ HSPC medium (SFEM II medium (STEMCELL Technologies) supplemented with 20 units/mL penicillin, 20 mg/mL streptomycin, Flt3-L (100 ng/mL), SCF (100 ng/mL), TPO (100 ng/mL), IL-6 (100 ng/mL), SR1 (0.75 μM) and UM171 (35 nM)) before being nucleoporated. Ribonucleoprotein (RNP) complexes were made by incubating Cas9 protein (Integrated DNA Technologies) with sgRNA at a molar ratio of 1:2.5 at 25° C. for 10 min prior to nucleoporation. Nucleoporation was performed using the Lonza 4D-Nucleofector™ System (program DZ100). After a 3-day recovery phase in CD34⁺ HSPC expansion medium, HSPC medium was changed to the medium promoting pDC differentiation as previously described.

### Donors

De-identified umbilical cord blood (UCB) samples were obtained following full-term caesarean sections deliveries of healthy infants at the Department of Gynecology and Obstetrics, Skejby Hospital, Aarhus.

### Statistical Analysis

All data were plotted using GraphPad Prism 6.0 (GraphPad Software, San Diego, Calif., USA). Data presented are expressed as means±standard error of mean (+/−SEM). Statistical analysis was performed using One-way ANOVA or two-way ANOVA, followed by Bonferroni post hoc test. Alpha=0.05.

### Example 1

### Production of pDCs

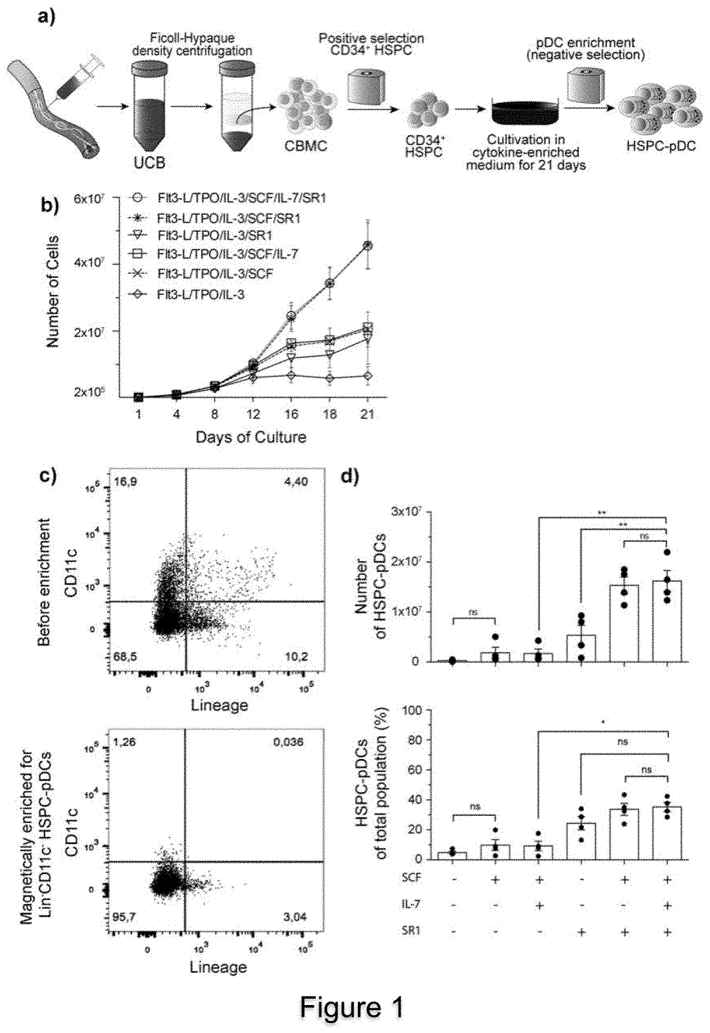

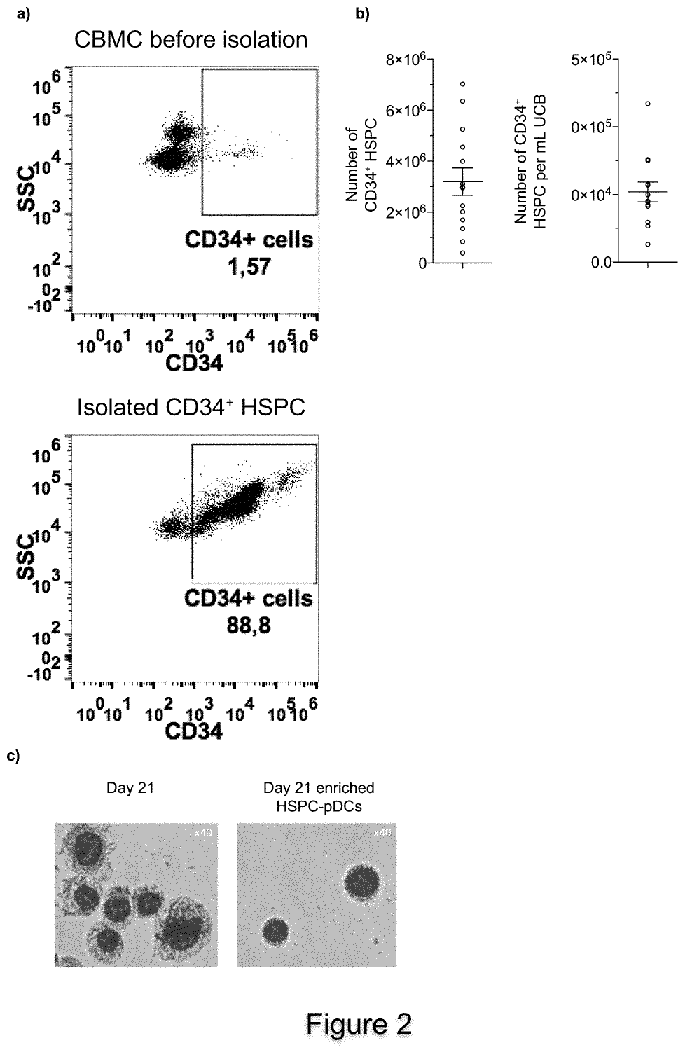

To validate the experimental approach, outcomes from the best practices were reproduced and a direct comparison was made [7-10] (FIG. 1a). As baseline condition, a previously reported pDC differentiation protocol entailing 21 days in medium containing the cytokines and growth factors Flt3 ligand (Flt3-L), thrombopoietin (TPO) and interleukin-3 was implemented [7]. Addition of stem cell factor (SCF) and StemRegenin 1 (SR1), resulted in a far greater expansion of progenitor cells (240 fold±42 compared to 35 fold±26 in baseline culture; FIG. 1b). Blood-derived pDCs lack the expression of lineage-specific surface markers (i.e. CD3, CD14, CD16, CD19, CD20 and CD56) and the conventional DC marker CD11c[1]. Therefore, to assess the number of putative pDCs defined as lin^{neg}CD11c^{neg} cells after the 21-day in vitro differentiation protocol, immunomagnetic negative selection were used to enrich for differentiated pDCs, hereafter referred to as HSPC-pDCs (FIG. 1c-d and FIG. 2a-c). SCF and SR1 proved to be indispensable additives to achieve high yield and purity of the HSPC-pDC population, with an average yield of 80 (±20) HSPC-pDC per single HSPC (corresponding to a total of 15×10⁶ HSPC-pDCs±4.2×10⁶ per 0.2×10⁶ HSPC). This is 57× higher than the 1.4 (±0.8) HSPC-pDCs per HSPC under baseline conditions.

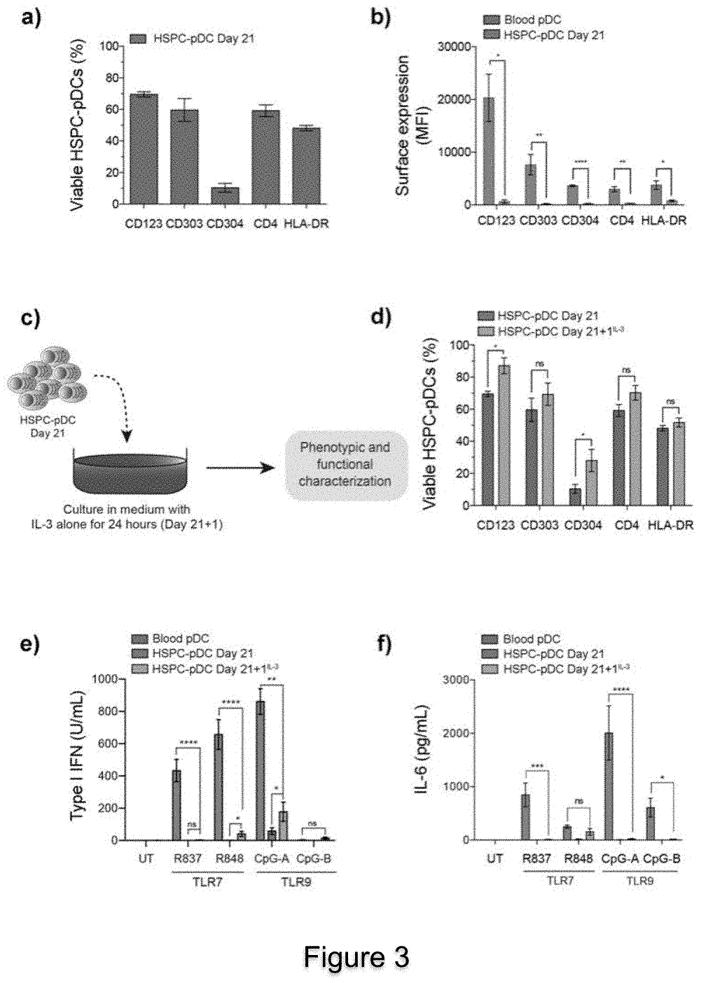

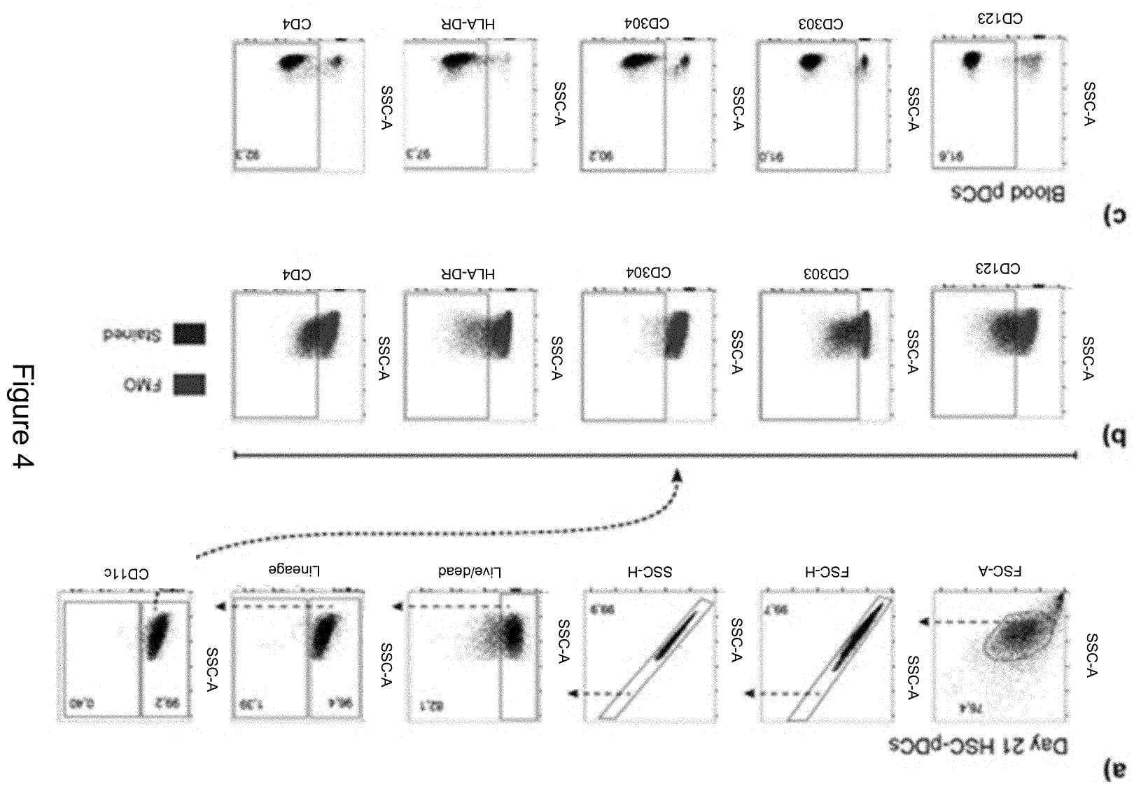

Next, it was examined whether the generated HSPC-pDCs were phenotypically and functionally equivalent to blood pDCs (FIG. 3a). Blood pDCs are characterized as being lin^{neg}CD11c^{neg}, while being positive for CD123 (IL3Rα), CD303 (BDCA2), CD304 (BDCA4), CD4 and HLA-DR [1, 13-15]. At culture day 21, 50-70% of HSPC-pDCs were positive for CD123, CD303, CD4 and HLA-DR, but only about 10% expressed CD304 (FIG. 3a and FIG. 4a-b). Furthermore, when compared to blood pDCs, HSPC-pDCs had significantly lower surface expression levels of the receptors as determined by the mean-fluorescent intensity (MFI) values (FIG. 3b and FIG. 4b-c).

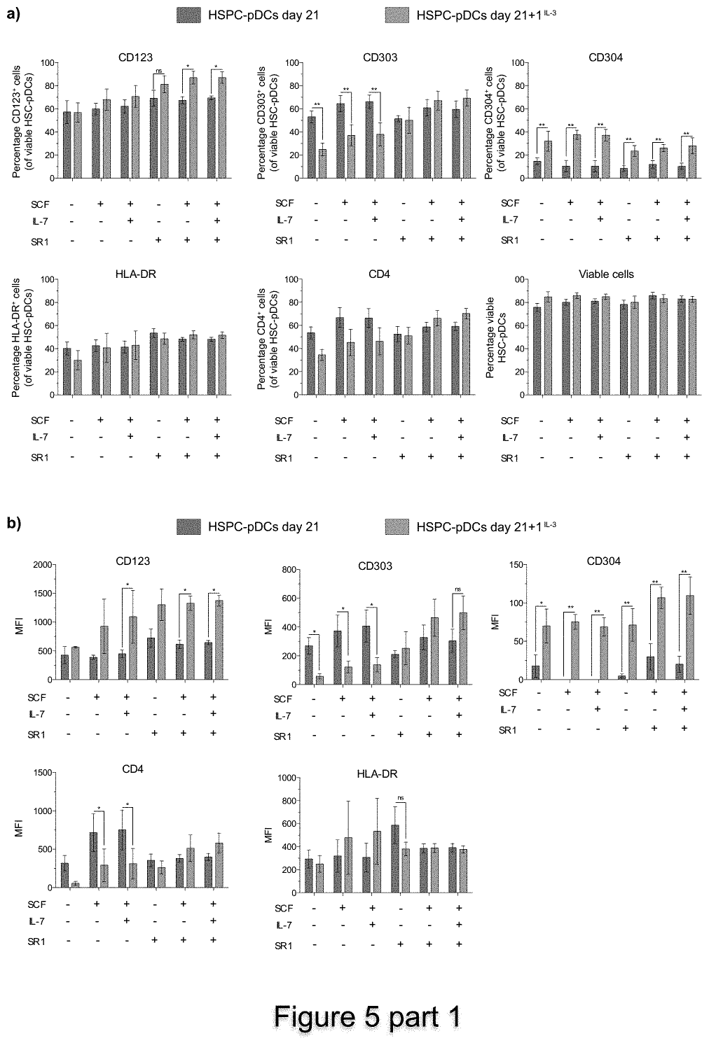

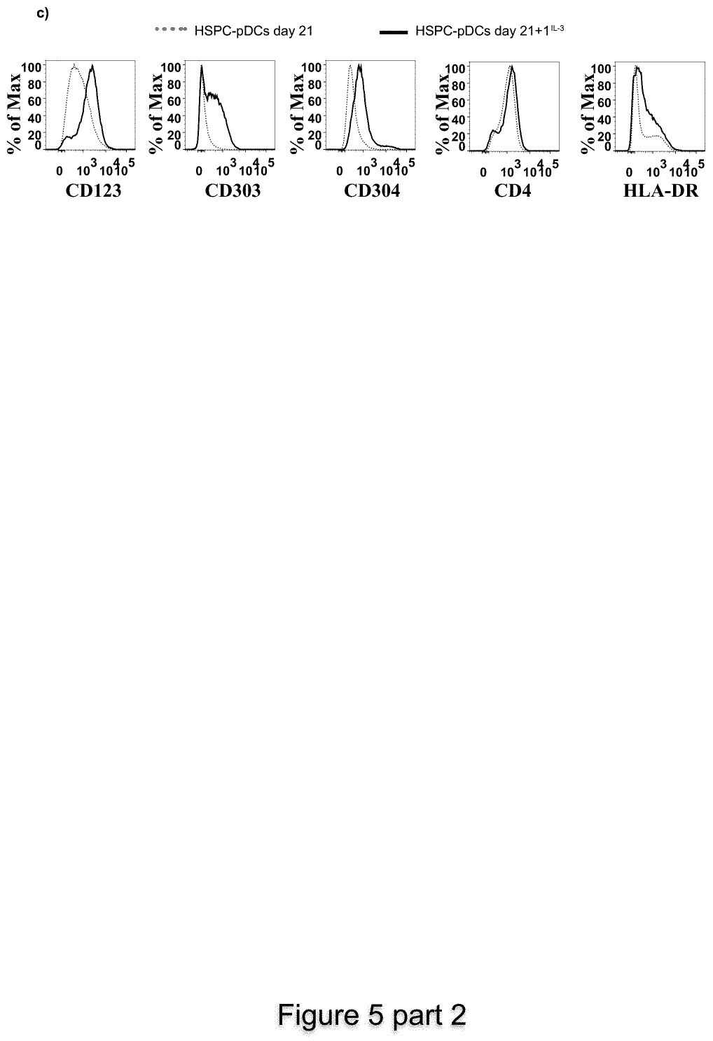

To test whether the differentiation medium arrested development of HSPC-pDCs, HSPC-pDCs were cultured for an additional 24 hours (Day 21+1) in medium supplemented only with IL-3 to support survival (FIG. 3c). Removal of growth factors led to a significant increase in surface expression of all the pDC-related markers compared to cells on day 21, but the emerging phenotype still did not fully recapitulate the phenotype of blood pDCs (FIG. 3d and FIG. 5a-c). Next, it was tested whether these HSPC-pDCs at day 21 or day 21+1 were able to respond to classical pDC activators (e.g. TLR7 and TLR9 agonists). Both cell populations were found to produce significantly less type I interferon and IL-6 compared to blood pDCs (FIG. 3e-f). The results show that at day 21 HSPC-pDCs are phenotypic and functional precursors to canonical pDCs, defining them as immature HSPC-pDCs.

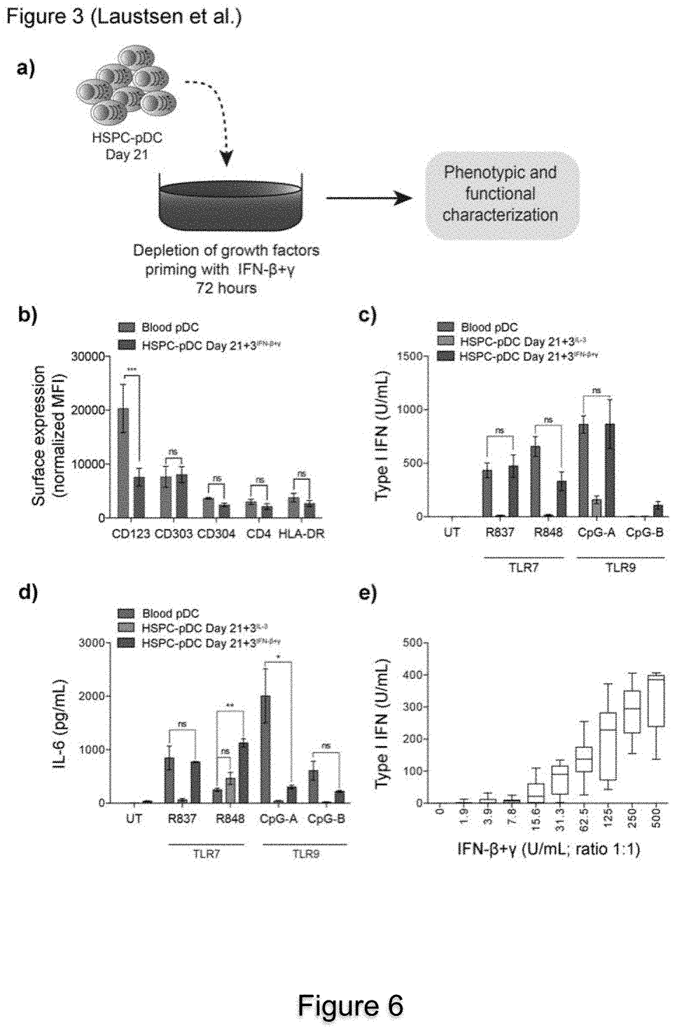

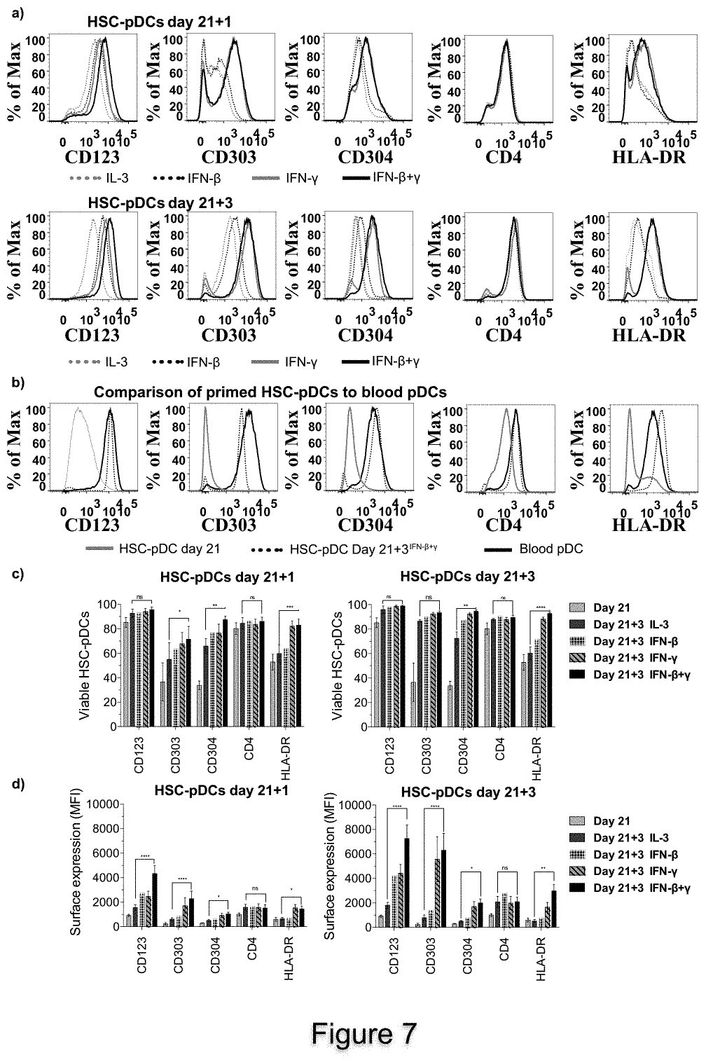

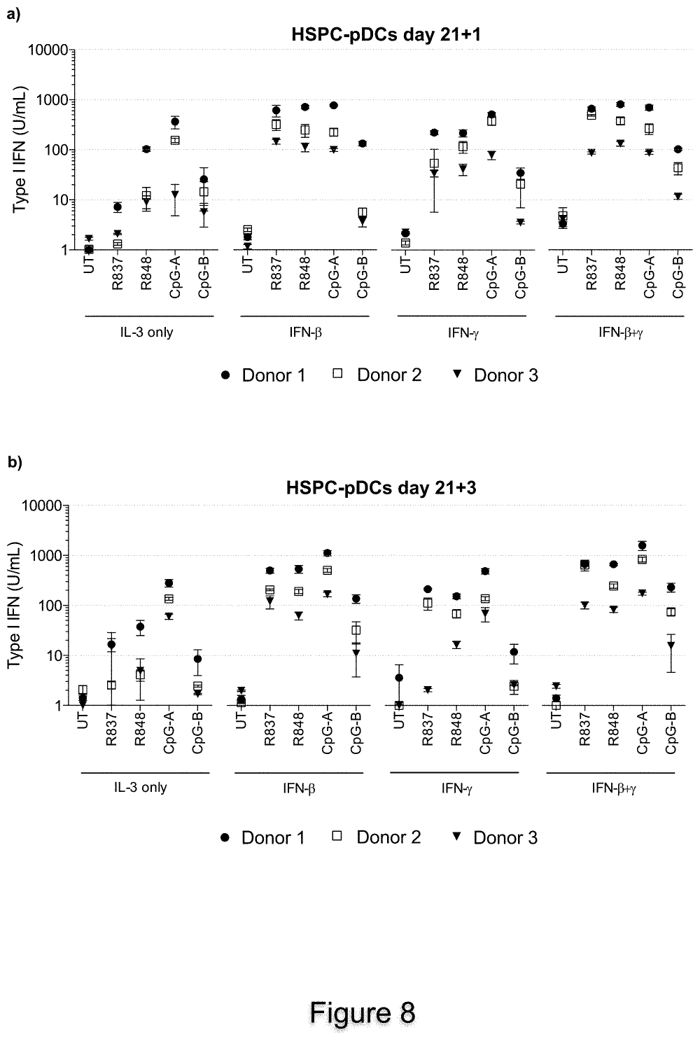

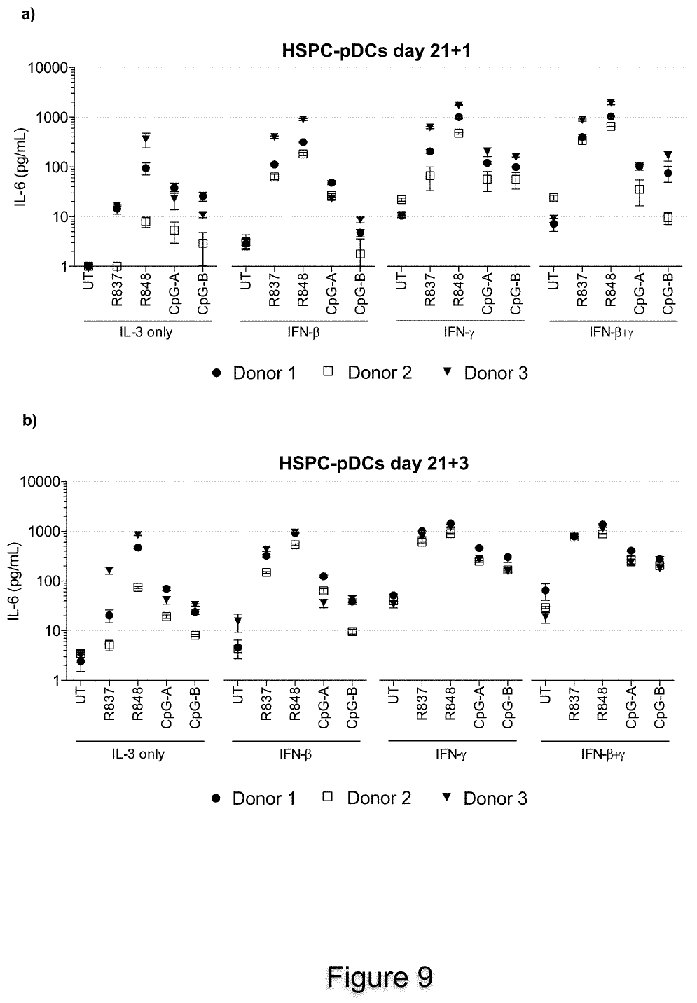

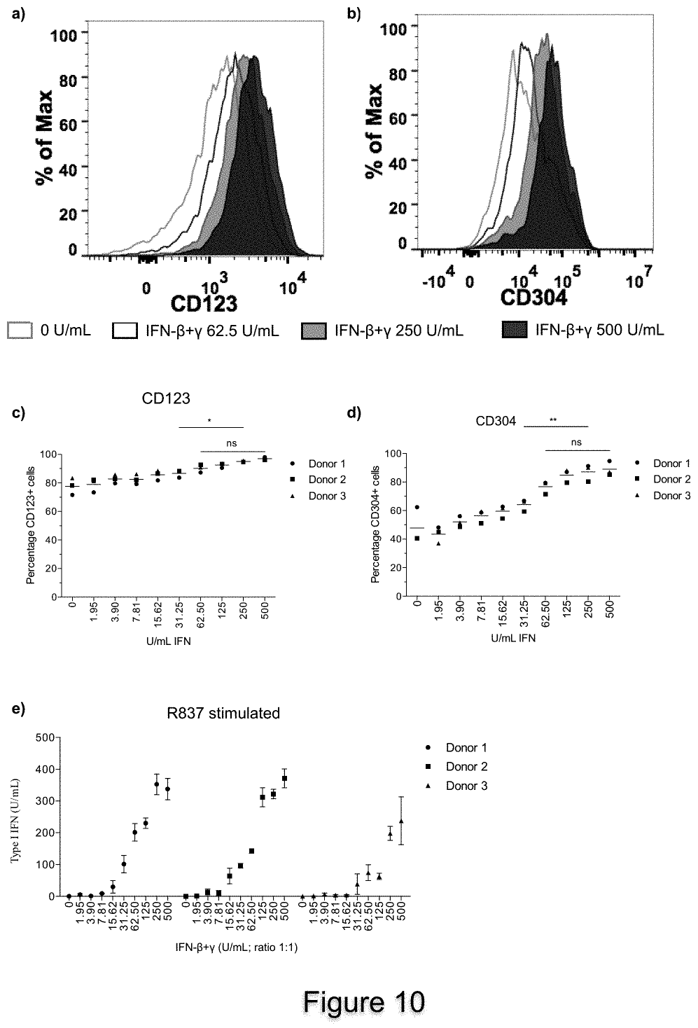

Subsequently, β- and/or γ-interferons (30 U/ml of each) together with IL-3 on Day 21 were added to the culture media for up to 72 hours (Day 21+1^{IFN-β+γ} and Day 21+3^{IFN-β+γ}) (FIG. 6a), and it was found that interferon primed HSPC-pDCs acquired a surface phenotype profile that strongly resembles blood pDCs with no significant differences in the expression of CD303, CD304, CD4 or HLA-DR (FIG. 6b and FIG. 7a-d). However, CD123 expression was still significantly lower, which has been reported to be caused by the supplement of IL-3 during culture pDCs [18]. Of note, the expression levels of CD303 and CD304 were improved by IFN-γ priming, which further increased by the addition of IFN-β (FIG. 7a-d). Next, Levels of functional type I IFN and IL-6 in response to TLR7 and TLR9 agonists of primed HSPC-pDCs were examined to test whether type I interferon and IL-6 secretion reached levels analogous to those produced by blood pDCs. Separately, IFN-γ predominantly promoted the secretion of IL-6, but was less effective at promoting a type I IFN response in comparison to IFN-β (FIG. 8a-b and 9a-b). Via joint titration of exogenous IFN-β and IFN-γ it was shown that HSPC-pDCs were able to acquire the phenotypic and functional traits of blood pDC, using as little as 30 U/ml each of recombinant IFN-β+γ for priming (FIG. 6c-e and FIG. 10a-e).

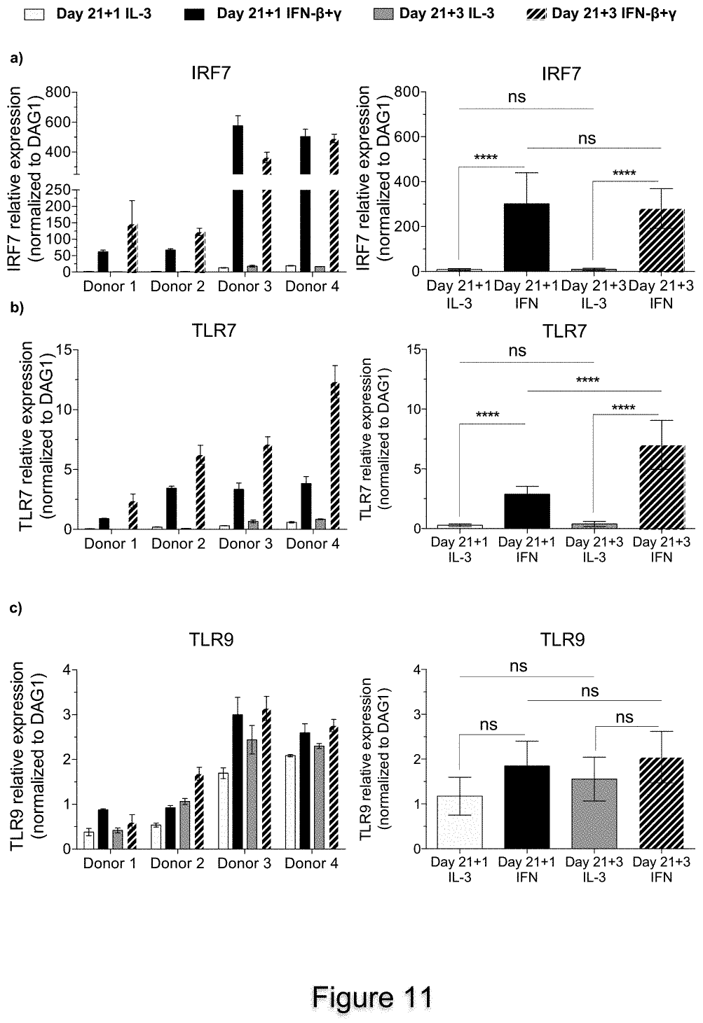

The expression levels of IRF7, TLR7 and TLR9 were examined by qPCR, and the results showed that interferon priming induced a significantly elevated expression of IRF7 and TLR7 (FIG. 11).

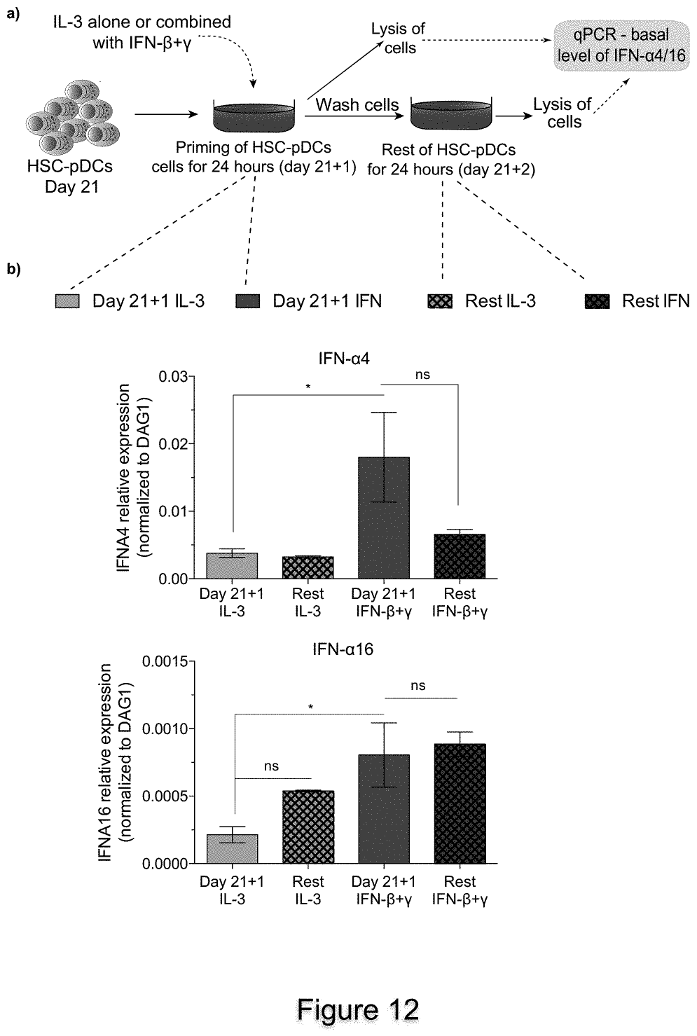

To test where exogenous priming of HSPC-pDCs initiated self-priming, IFN-β and IFN-γ were removed from HSPC-pDCs after one day in culture, and let the cells rest for an additional day in medium with IL-3 alone (FIG. 12a). Overall, priming of HSPC-pDCs led to increased expression of the two IFN-α mRNA isotypes tested (α4, α16). After the resting phase HSPC-pDCs still expressed IFN-α, indicating self-priming capacities within the culture (FIG. 12a-b).

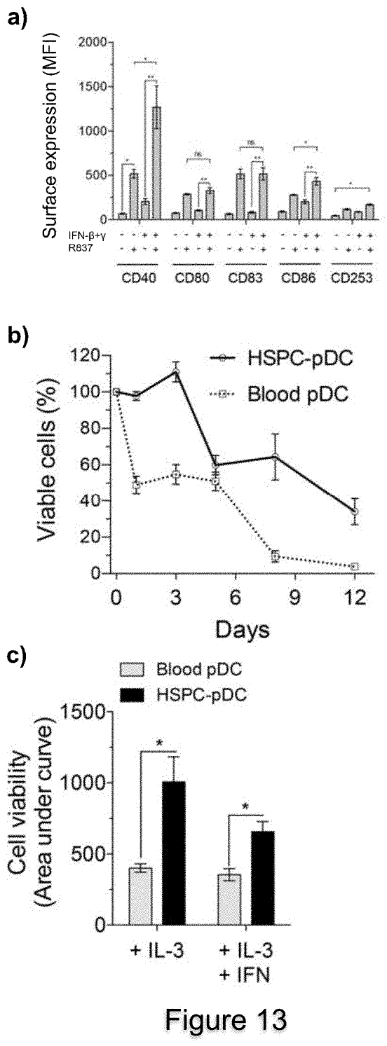

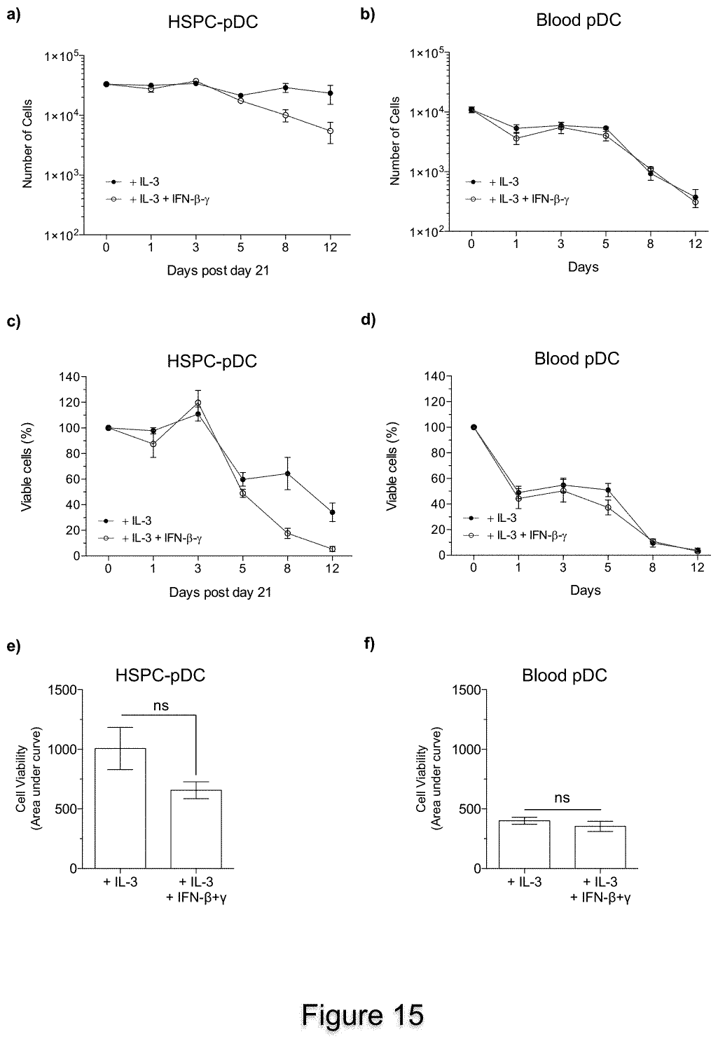

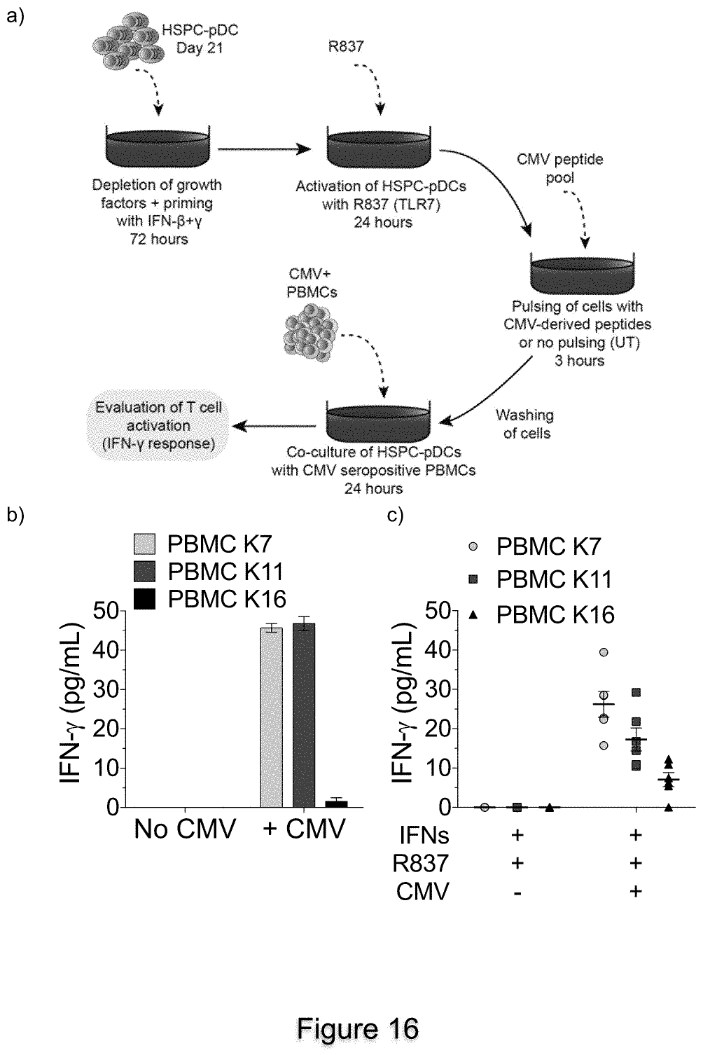

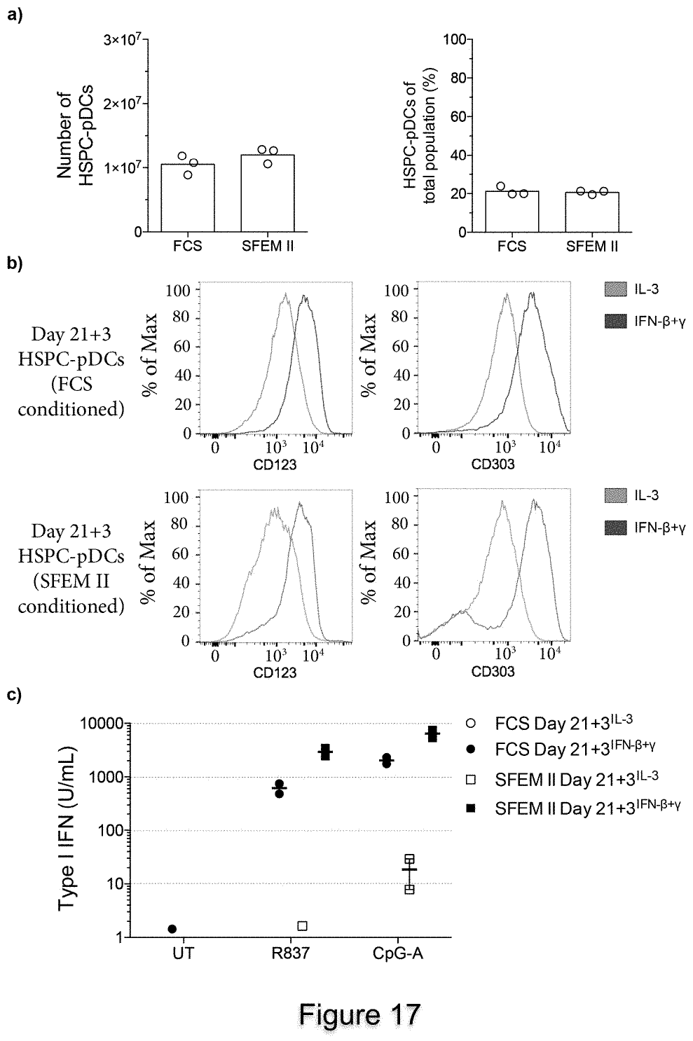

One of the main drawbacks of using blood pDCs is their short lifespan in culture, limiting the window of opportunity for experimental use. We therefore investigated the viability of HSPC-pDCs to evaluate the use of this model in prolonged culture studies. We compared the survival of HSPC-pDCs post day 21 and blood pDCs over a prolonged culture of 12 days in medium with IL-3 and +/−IFN-β+γ. 50% of all blood pDCs were found to die within 3 days post isolation, whereas HSPC-pDCs demonstrated superior survival during this time frame (FIG. 13b and FIG. 15a-f). After 5 days of culture, the viability of HSPC-pDCs dropped to 60%, similar to blood-pDCs. Notably, after 8 days of culture, all blood pDCs were dead, whereas 60% of the HSPC-pDCs remained viable (FIG. 13b-c and FIG. 15a-f). After 12 days in culture HSPC-pDC still demonstrated more than 40% survival.

Taken together, our data illustrate that replacing “sternness maintaining” culture factors with interferons on culture day 21 promote the differentiation of pDC-precursor cells into bona fide pDCs with strong type I interferon production and survival capacity.

### Example 2

### Production of APCs for Therapeutic Use

In addition to producing Type I IFN, pDCs can function as antigen presenting cells (APCs). One approach of using our technology is within anti-tumor immuno-therapy. Hematopoietic stem and progenitor cells (HSPC) will first be extracted from a blood sample from a patient. The HSPCs will then be cultured under specific conditions to become pDCs (HSPC-pDC). HSPC-pDCs will then be primed to reach maturity. Primed HSPC-pDC will then be activated with an inactive viral vaccine strain FSME, while simultaneously being loaded with tumour antigens. This will induce an antigen-presenting phenotype in HSPC-pDC. Tumour antigens can be acquired from the cancer cells directly or indirectly, depending on what is known about the specific type of cancer. Directly acquisition entails loading of specific known tumor antigens onto the pDCs through the use of liposome nano-carriers. Indirect loading involves isolation of cancer cells and co-culture of irradiated cancer cells with FSME-activated pDCs, resulting in attachment of cancer cell membrane to the surface of pDCs, which then can be presented to T and B cells. Both methods will induce an antigen-presenting phenotype in HSPC-pDC. Activated and antigen-loaded HSPC-pDC can then be re-introduced into the patient to leading to:
1. Direct inhibition of growth and induction of cancer cell death through killer-pDC activity.
2. Activation of surrounding immune cells in the tumour area, including natural killer cells.
3. Activation of antigen-specific B cells and cytotoxic T cells.

Overall, this will innate a cascade that kill cancer cells, and establish a long-lasting anti-tumoral response in the patient so timor-relapse is averted.

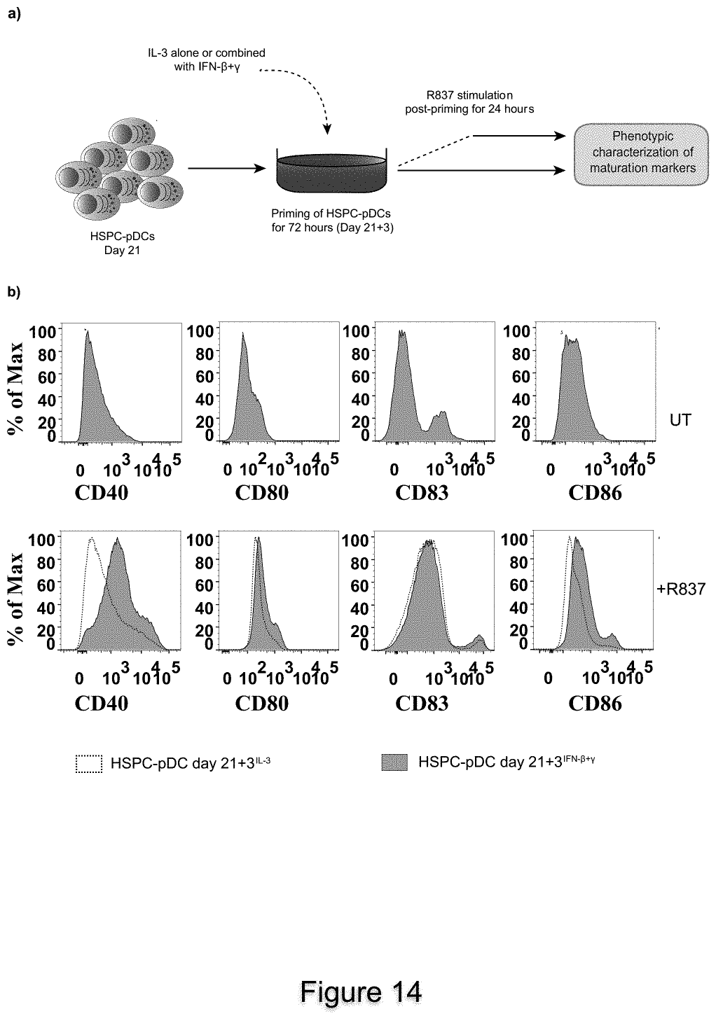

To test whether our IFN-primed HSPC-pDCs exhibited an APC phenotype we evaluated the expression of classical APC activation markers (CD40, CD80, CD83, and CD86). Non-primed as well as IFN-β+γ primed HSPC-pDCs treated with TLR7 agonist displayed elevated expression of CD80 and CD83 (FIG. 13a and FIG. 14a-b). In contrast, expression of CD40 and CD86 was significantly higher in IFN-β+γ primed cells compared to non-primed. To extend our observations, we investigated if HSPC-pDCs were capable of activating antigen-specific T cells. To determine this we loaded primed and TLR7 activated HSPC-pDCs with peptides derived from CMV and subsequently co-cultured them with PBMCs from CMV sero-positive donors. Activation of CMV+ T cells was higher in CMV loaded HSPC-pDCs than to HSPC-pDCs without exogenous protein as evidenced by the IFN-γ response. Together, we interpret these data to indicate that primed and activated HSPC-pDCs effectively present and activate antigen-specific T cells

### Example 3

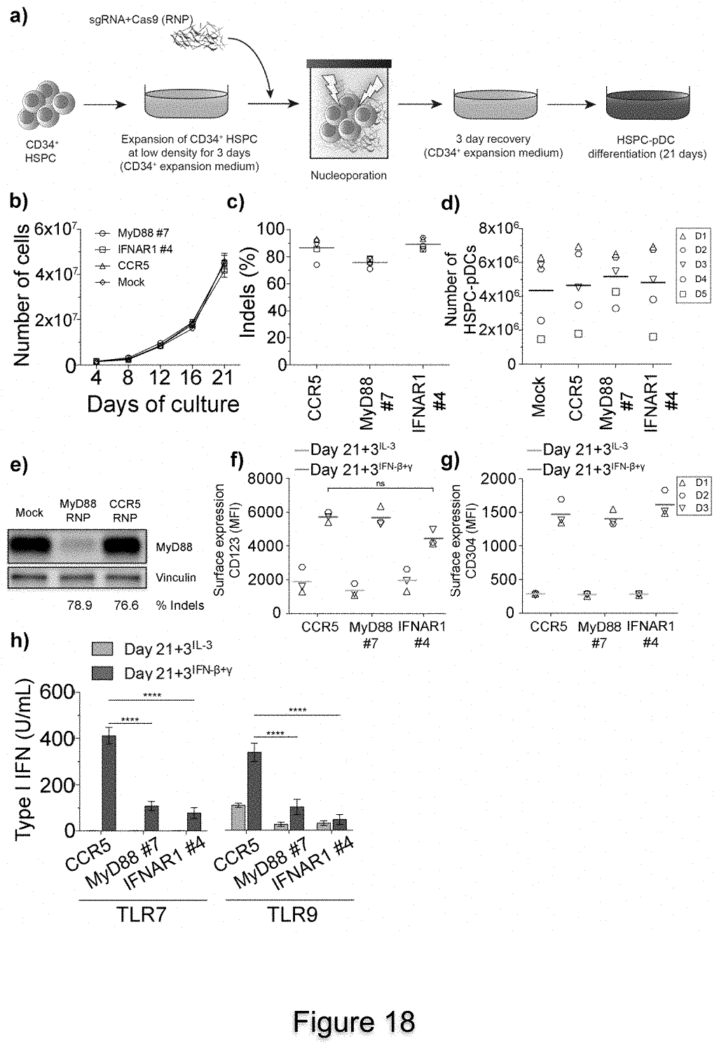

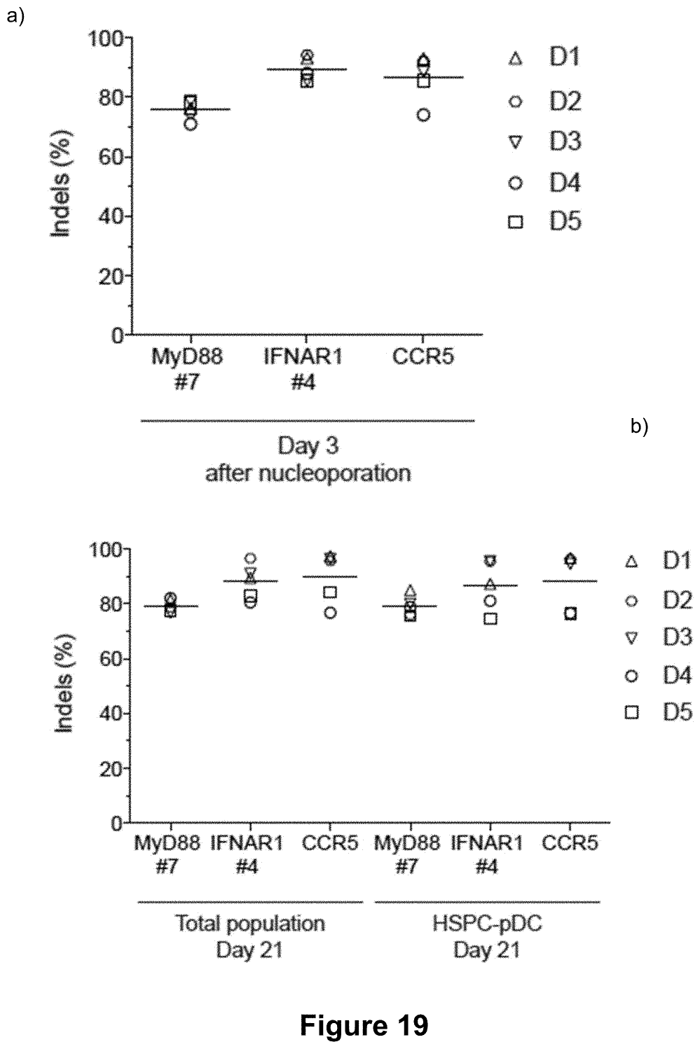

### Production of Genetically Modified HSPC-pDCs

Whereas genetic manipulation of pDCs is currently impossible, recent advances in the CRISPR/Cas9 technology have resulted in successful and efficient gene editing of CD34⁺ HSPCs. Thus, we speculated that gene-edited pDCs could be generated successfully by initial modification of CD34⁺ HSPCs and subsequent differentiation into pDCs. We employed a gene editing strategy where synthetic chemically modified sgRNAs and recombinant Cas9 protein, which forms a ribonucleoprotein (RNP) complex, were delivered to CD34⁺ HSPCs by electroporation (FIG. 18a). The sgRNAs were designed to target the open reading frame within the first exon of IFNAR1 (subunit of the type I IFN receptor) and MyD88. IFNAR1 was selected owing to its role in TLR-mediated responses, whereas MyD88 was included for its established role in TLR-mediated responses. A previously published sgRNA targeting the safe harbor gene CCR5 was used as negative control. A range of sgRNAs for MyD88 and IFNAR1 were initially screened for their potency to induce targeted gene disruption by insertions and deletions (Indels) by plasmid electroporation in K562 cells. The three sgRNAs with the highest efficacy for each target were then tested as synthetic sgRNAs with chemically modified nucleotides at both termini by Cas9 RNP delivery by electroporation of CD34⁺ HSPCs, leading to the selection of a single potent sgRNA for each target gene yielding 73.4%±4.5% and 86.7%±2.7% Indel frequencies for MyD88 and IFNAR1, respectively (FIG. 19a). The two selected sgRNAs and the CCR5 control sgRNA were then used in CD34⁺ HSPCs that following a 3-day recovery phase after electroporation were differentiated into pDCs (FIG. 18a). Importantly, no change in proliferation rate and total cell yield was observed during differentiation and high Indel rates were observed pre- and post-differentiation (FIG. 18b-c). When we evaluated the overall yield of HSPC-pDCs on day 21 we observed a 4-fold decrease in yield compared to previous experiments. We ascribed this to either the nucleoporation procedure itself, as mock-electroporated cells were equally affected, or the increased period of cultivation (FIG. 18d). Analysis of Indel frequencies after immunomagnetic pDC enrichment confirmed that HSPC-pDCs had similar levels of Indels as the total non-enriched cell population (FIG. 19), and MyD88 protein levels determined by Western blotting confirmed potent knock-down (FIG. 18e). Next, HSPC-pDCs were analyzed for two pDC phenotypic markers (CD123 and CD304) and for functionality succeeding IFN priming. sgRNA-targeted cells showed high phenotypic expression of both surface markers and following IFNalpha receptor 1 and MyD88-targeted HSPC-pDCs were then stimulated with TLR7/9 agonists in the absence and presence of IFN priming. In both cases, type I IFN induction was completely absent in the KO cells (FIG. 18h). Interestingly, when we evaluated the response to TLR agonists in HSPC-pDCs targeted at the IFNAR1 gene, we found that IFNAR1-depleted cells displayed a complete lack of type I IFN induction, supporting our previous findings that type I IFN feedback is a prerequisite for TLR7 and TLR9-dependent type I IFN induction (FIG. 18h). In summary, we demonstrate that gene-edited HSPC-pDCs can be produced by initially modifying CD34⁺ HSPC before differentiation to HSPC-pDCs. Using this system, we confirm that while MyD88 and IFNAR1 do not appear to be implicated in human pre-pDC development, they are essential for functional pDC immune responses upon stimulation through the TLR pathway.

### Example 4

### Production of Genetically Modified HSPC-pDCs With Improved Therapeutic Capacity

Hematopoietic stem and progenitor cells (HSPC) will first be extracted from a blood sample from a patient. The HSPCs will then be cultured under specific conditions to make easier for a gene editing procedure. For the gene editing procedure, a ribonucleoprotein (RNP) complex consisting of a Cas9 protein and a sgRNA targeting the gene of interest will be delivered to HSPCs using electroporation. After the gene editing procedure HSPCs will be cultured under specific conditions to become pDCs (HSPC-pDCs). The efficiency of the gene editing procedure can be estimated through PCR (TIDE analysis) and by measuring phenotypic and functional responses (e.g. protein levels using western blot). Overall, this procedure will generate non-homologous end-joining (NHEJ), leading to breaks in the DNA of the gene of interest, thus generating HSPC-pDCs where a gene of interest has been removed (knocked-out). In another procedure, adeno-associated virus (AAV) can be used to deliver a repair template to the HSPCs, permitting homologous recombination to occur, leading to the generation of HSPC-pDCs that have edited genetically. Overall, these procedure will allow:
1. Investigation of specific molecular pathways in pDCs by removing a gene of interest
2. Increase the therapeutic potential of HSPC-pDCs by:
   a. Removing receptors that are known to inhibit the function of pDCs in cancer, including PD-L1 and CD85g.
   b. Increasing the anti-tumoral potential of HSPC-pDCs by increasing levels of TRAIL on the surface of the cell

The present invention provides a powerful strategy for expanding and differentiating human HSPC into pDCs that overcomes the great need for a robust ex vivo pDC culture system to gain clear insights into pDC biology. The power in this model is found within its simplicity, the increased survival of the pDC and the high cell yields, which means that this approach is readily amenable to investigation-specific variables and modifications.

### REFERENCES

1. Swiecki, M. and M. Colonna, The multifaceted biology of plasmacytoid dendritic cells. Nat Rev Immunol, 2015. 15(8): p. 471-85.
2. Tang, M., J. Diao, and M. S. Cattral, Molecular mechanisms involved in dendritic cell dysfunction in cancer. Cell Mol Life Sci, 2016.
3. Tovey, M. G., C. Lallemand, and G. Thyphronitis, Adjuvant activity of type I interferons. Biol Chem, 2008. 389(5): p. 541-5.
4. Rajagopal, D., C. Paturel, Y. Morel, S. Uematsu, S. Akira, and S. S. Diebold, Plasmacytoid dendritic cell-derived type I interferon is crucial for the adjuvant activity of Toll-like receptor 7 agonists. Blood, 2010. 115(10): p. 1949-57.
5. Ueda, Y., M. Hagihara, A. Okamoto, A. Higuchi, A. Tanabe, K. Hirabayashi, S. Izumi, T. Makino, S. Kato, and T. Hotta, Frequencies of dendritic cells (myeloid DC and plasmacytoid DC) and their ratio reduced in pregnant women: comparison with umbilical cord blood and normal healthy adults. Hum Immunol, 2003. 64(12): p. 1144-51.
6. Zhan, Y., K. V. Chow, P. Soo, Z. Xu, J. L. Brady, K. E. Lawlor, S. L. Masters, M. O'Keeffe, K. Shortman, J. G. Zhang, and A. M. Lew, Plasmacytoid dendritic cells are short-lived: reappraising the influence of migration, genetic factors and activation on estimation of lifespan. Sci Rep, 2016. 6: p. 25060.
7. Demoulin, S., P. Roncarati, P. Delvenne, and P. Hubert, Production of large numbers of plasmacytoid dendritic cells with functional activities from CD34(+) hematopoietic progenitor cells: use of interleukin-3. Exp Hematol, 2012. 40(4): p. 268-78.
8. Olivier, A., E. Lauret, P. Gonin, and A. Galy, The Notch ligand delta-1 is a hematopoietic development cofactor for plasmacytoid dendritic cells. Blood, 2006. 107(7): p. 2694-701.
9. Curti, A., M. Fogli, M. Ratta, S. Tura, and R. M. Lemoli, Stem cell factor and FLT3-ligand are strictly required to sustain the long-term expansion of primitive CD34+DR− dendritic cell precursors. J Immunol, 2001. 166(2): p. 848-54.
10. Thordardottir, S., B. N. Hangalapura, T. Hutten, M. Cossu, J. Spanholtz, N. Schaap, T. R. Radstake, R. van der Voort, and H. Dolstra, The aryl hydrocarbon receptor antagonist StemRegenin 1 promotes human plasmacytoid and myeloid dendritic cell development from CD34+ hematopoietic progenitor cells. Stem Cells Dev, 2014. 23(9): p. 955-67.
11. Hoffman, R., J. Tong, J. Brandt, C. Traycoff, E. Bruno, B. W. McGuire, M. S. Gordon, I. McNiece, and E. F. Srour, The in vitro and in vivo effects of stem cell factor on human hematopoiesis. Stem Cells, 1993. 11 Suppl 2: p. 76-82.
12. Boitano, A. E., J. Wang, R. Romeo, L. C. Bouchez, A. E. Parker, S. E. Sutton, J. R. Walker, C. A. Flaveny, G. H. Perdew, M. S. Denison, P. G. Schultz, and M. P. Cooke, Aryl hydrocarbon receptor antagonists promote the expansion of human hematopoietic stem cells. Science, 2010. 329(5997): p. 1345-8.
13. Siegal, F. P., N. Kadowaki, M. Shodell, P. A. Fitzgerald-Bocarsly, K. Shah, S. Ho, S. Antonenko, and Y. J. Liu, The nature of the principal type 1 interferon-producing cells in human blood. Science, 1999. 284(5421): p. 1835-7.
14. Cella, M., D. Jarrossay, F. Facchetti, O. Alebardi, H. Nakajima, A. Lanzavecchia, and M. Colonna, Plasmacytoid monocytes migrate to inflamed lymph nodes and produce large amounts of type I interferon. Nat Med, 1999. 5(8): p. 919-23.
15. Dzionek, A., A. Fuchs, P. Schmidt, S. Cremer, M. Zysk, S. Miltenyi, D. W. Buck, and J. Schmitz, BDCA-2, BDCA-3, and BDCA-4: three markers for distinct subsets of dendritic cells in human peripheral blood. J Immunol, 2000. 165(11): p. 6037-46.
16. Chen, Y. L., T. T. Chen, L. M. Pai, J. Wesoly, H. A. Bluyssen, and C. K. Lee, A type I IFN-Flt3 ligand axis augments plasmacytoid dendritic cell development from common lymphoid progenitors. J Exp Med, 2013. 210(12): p. 2515-22.
17. Kim, S., V. Kaiser, E. Beier, M. Bechheim, M. Guenthner-Biller, A. Ablasser, M. Berger, S. Endres, G. Hartmann, and V. Hornung, Self-priming determines high type I IFN production by plasmacytoid dendritic cells. Eur J Immunol, 2014. 44(3): p. 807-18.
18. Schmitt, N., M. C. Cumont, M. T. Nugeyre, B. Hurtrel, F. Barre-Sinoussi, D. Scott-Algara, and N. Israel, Ex vivo characterization of human thymic dendritic cell subsets. Immunobiology, 2007. 212(3): p. 167-77.
19. Mauriz, J. L. and J. Gonzalez-Gallego, Antiangiogenic drugs: current knowledge and new approaches to cancer therapy. J Pharm Sci, 2008. 97(10): p. 4129-54.
20. Gold, M. C., E. Donnelly, M. S. Cook, C. M. Leclair, and D. A. Lewinsohn, Purified neonatal plasmacytoid dendritic cells overcome intrinsic maturation defect with TLR agonist stimulation. Pediatr Res, 2006. 60(1): p. 34-7.
21. Schuller, S. S., K. Sadeghi, L. Wisgrill, A. Dangl, S. C. Diesner, A. R. Prusa, K. Klebermasz-Schrehof, S. Greber-Platzer, J. Neumuller, H. Helmer, P. Husslein, A. Pollak, A. Spittler, and E. Forster-Waldl, Preterm neonates display altered plasmacytoid dendritic cell function and morphology. J Leukoc Biol, 2013. 93(5): p. 781-8.
22. De Wit, D., V. Olislagers, S. Goriely, F. Vermeulen, H. Wagner, M. Goldman, and F. Willems, Blood plasmacytoid dendritic cell responses to CpG oligodeoxynucleotides are impaired in human newborns. Blood, 2004. 103(3): p. 1030-2.

The following items represent preferred embodiments of the present invention.

### Items

1. A method for producing a plasmacytoid dendritic cells (pDCs), said method comprising
providing hematopoietic stem progenitor cells (HSPCs)
incubating said HSPCs in a first medium comprising cytokines and growth factor whereby said HSPCs are differentiated into precursor-pDCs
adding interferons (IFNs) to said first medium to obtain a second medium
   whereby said precursor-pDCs are differentiated into pDCs
   2. The method according to item 1, wherein said second medium comprises IFN-γ and/or IFN-γ.
   3. The method according to any of items 1 and 2, wherein said second medium comprises IL-3.
   4. The method according to any of the preceding items, wherein said precursor-pDCs are incubated for at least 24 hours in said second medium.
   5. The method according to any of the preceding items, wherein said precursor-pDCs are incubated for 24 to 72 hours in said second medium.
   6. The method according to any of the preceding items, wherein said first medium comprises Flt3 ligand, thrombopoietin and/or interleukin-3.
   7. The method according to any of the preceding items, wherein said first medium comprises stem cell factor and StemRegenin 1.
   8. The method according to any of the preceding items, wherein said HSPCs are incubated for 21 days in said first medium.
   9. The method according to any of the preceding items, wherein said first medium comprises UM 171.
   10. The method according to any of the preceding items, wherein incubation of said HSPCs in said first medium lead to an average yield of 60-100 precursor-pDCs per HPC.
   11. The method according to any of the preceding items, further comprising a step of immunomagnetic negative selection to enrich for differentiated pDCs.
   12. The method according to any of the preceding items, further comprising a step of incubating said pDCs with at least one antigen leading to the formation of antigen presenting cells (APCs).
   13. The method according to item 12, wherein said antigen is a cancer specific antigen.
   14. The method according to item 12, wherein said antigen is a pathogen specific antigen.
   15. The method according to item 12, wherein said antigen is a virus-, bacteria- or parasite specific antigen.
   16. The method according to any of the preceding items, wherein said pDCs express TRAIL.
   17. The method according to any of the preceding items, wherein said pDCs express CD123, CD303, CD304, CD4 and/or HLA-DR.
   18. The method according to any of the preceding items, wherein said pDCs express IFN type I, IFN type II, IFN type III and/or proinflammatory cytokines.
   19. The method according to any of the preceding items, wherein said pDCs express IRF7, TLR7 and/or TLR9.
   20. The method according to any of the preceding items, wherein said pDCs express CD40, CD80, CD83 and/or CD86.
   21. The method according to any of the preceding items, wherein said pDCs secretes IL-6.
   22. A pDC and/or an APC population obtained by the method according to any of the preceding items.
   23. The pDC and/or APC population according to item 22, wherein said pDCs and/or APCs are as defined in any of items 16 to 21.
   24. A pDC and/or APC population as defined in any of items 22 to 23 for use in the treatment of an infectious disease or cancer.
   25. A pharmaceutical formulation, which comprises a pDC and/or an APC population according to any of items 22 to 23 and a pharmaceutically acceptable carrier.
   26. A vaccine comprising an immunologically effective amount of a pDC or an APC population according to any of items 22 to 23.
   27. A kit comprising a pDC or an APC population according to any of items 22 to 23, and elements associated with immunological use.
   28. The kit according to item 27, further comprising at least one antigen.
   29. The kit according to item 28, wherein said antigen is as defined in any of items 13 to 15.
   30. A method of treating, preventing and/or ameliorating an infectious disease and/or cancer, said method comprising administering a therapeutically efficient amount of a pDC or an APC population according to any of items 22 to 23 to an individual in need thereof.
   31. The method according to item 30, wherein said infectious disease is a viral infection or a bacterial infection.
   32. The method according to item 30, wherein said cancer is TRAIL specific cancer.
   33. The method according to any of items 30 to 32, wherein said pDC and/or APC population is/are administered to the individual in need thereof by injection.

1. A method for producing a plasmacytoid dendritic cells (pDCs), said method comprising
providing hematopoietic stem progenitor cells (HSPCs)
incubating said HSPCs in a first medium comprising cytokines and growth factor whereby said HSPCs are differentiated into precursor-pDCs
adding SCF and SR1 in a first medium to obtain high yield of pre-cursor pDCs
providing a second medium comprising interferons (IFNs)
adding said second medium to said first medium comprising pre-cursor pDCs, whereby said precursor-pDCs are transformed into activated and differentiated pDCs

2. The method according to claim 1, wherein said second medium comprises IFN-γ and/or IFN-β.

3. The method according to any of claims 1 and 2, wherein said second medium comprises IL-3.

4. The method according to any of the preceding claims, wherein said first medium comprises Flt3 ligand, thrombopoietin and/or interleukin-3.

5. The method according to any of the preceding claims, wherein said first medium comprises stem cell factor and StemRegenin 1.

6. The method according to any of the preceding claims, wherein said first medium comprises UM 171.

7. The method according to any of the preceding items, wherein said HSPCs are incubated for 21 days in said first medium.

8. The method according to any of the preceding items, wherein said precursor-pDCs are incubated for at least 24 hours in said second medium.

9. The method according to any of the preceding items, wherein said precursor-pDCs are incubated for 24 to 72 hours in said second medium.

10. The method according to any of the preceding claims, wherein said HSPCs are genetically modified.

11. The method according to claim 7, wherein said HSPCs are genetically modified using delivery of single guide RNA (sgRNA) and/or adeno-associated virus using electroporation and/or transduction

12. The method according to claim 7 or 11, wherein said HSPCs has been genetically modified by knock-out of one or more co-stimulatory factors

13. The method according to claim 12, wherein said one or more co-stimulatory factors are selected from the group consisting of the receptors for PGE2 and TGF-β, as well as CD85g, IDO, ICOS-L and PD-L1.

14. The method according to any of the preceding claims, wherein incubation of said HSPCs in said first medium lead to an average yield of 60-100 precursor-pDCs per HPC.

15. The method according to any of the preceding claims, further comprising a step of immunomagnetic negative selection to enrich for differentiated pDCs.

16. The method according to any of the preceding claims, further comprising a step of incubating incubating said pDCs with at least one antigen leading to the formation of antigen presenting cells (APCs).

17. The method according to any of the preceding claims, wherein said pDCs express TRAIL.

18. The method according to any of the preceding claims, wherein said pDCs express CD123, CD303, CD304, CD4 and/or HLA-DR.

19. The method according to any of the preceding items, wherein said pDCs express IFN type I, IFN type II, IFN type III and/or proinflammatory cytokines.

20. The method according to any of the preceding items, wherein said pDCs express IRF7, TLR7 and/or TLR9.

21. A pDC and/or an APC population obtained by the method according to any of the preceding claims.

22. A pharmaceutical formulation, which comprises a pDC and/or an APC population according to claim 21 and a pharmaceutically acceptable carrier.

23. A vaccine comprising an immunologically effective amount of a pDC or an APC population according to claim 21.

24. A pDC and/or APC population as defined in claim 21 and/or a pharmaceutical formulation as defined in claim 22 and/or a pharmaceutical formulation as defined in claim 23 for use in the treatment of an infectious disease or cancer.

25. A method of treating an infectious disease or cancer, said method comprising administering a therapeutically efficient amount a pDC and/or APC population as defined in claim 21 and/or a pharmaceutical formulation as defined in claim 22 and/or a vaccine as defined in claim 23 to a subject in need thereof.

26. The method according to item 25, wherein said infectious disease is a viral infection or a bacterial infection.

27. The method according to item 25, wherein said cancer is TRAIL specific cancer.

28. The method according to any of items 25 to 27, wherein said pDC and/or APC population is/are administered to the individual in need thereof by injection.
